(19) United States
(12) Patent Application Publication
(10) Pub. No.: US 2005/0032243 A1
(43) Pub. Date: Feb. 10, 2005

(54) Method and apparatus for rapid determination of ligand-protein binding using charcoal adsorption

(76) Inventor: Gary M. Pollack, Chapel Hill, NC (US)

Correspondence Address: JENKINS & WILSON, PA, 3100 TOWER BLVD, SUITE 1400, DURHAM, NC, 27707, US

(21) Appl. No.: 10/484,676

(22) PCT Filed: Jul. 25, 2002
(86) PCT No.: PCT/US02/23556

Related U.S. Application Data
(60) Provisional application No. 60/307,732, filed on Jul. 25, 2001.

(51) Int. Cl. G01N 33/543

(52) U.S. Cl. 436/518

(57) ABSTRACT

A method for evaluating binding of a ligand to a target protein. The method includes the steps of: (a) providing a sample comprising a target protein and a ligand, wherein the target protein and ligand are suspected to be bound reversibly together in a complex; (b) preconditioning activated charcoal with the target protein; (c) contacting the sample with the preconditioned activated charcoal for a time sufficient to allow for adsorption of unbound ligand to the activated charcoal; (d) eluting the sample from the activated charcoal; and (e) determining an amount of ligand in the eluted sample to thereby evaluate binding of the ligand to the target protein. An apparatus useful in carrying out the method, and a method of making the same, are also disclosed.

### RELATED APPLICATIONS

This application claims the benefit of U.S. provisional patent application No. 60/307,732, filed Jul. 25, 2001, the disclosure of which is incorporated herein by reference in its entirety.

### FIELD OF THE INVENTION

The present invention generally relates to methods for measuring protein-ligand interactions. More particularly, the present invention provides an apparatus and method for determining unbound ligand fraction in the presence of a target protein, such as a plasma protein.

Table of Abbreviations

| | |
|---|---|
| BSA | bovine serum albumin |
| CPM | counts per minute |
| DCC | dextran-coated charcoal |
| DIGT | digitoxin |
| DPH | diphenylhydantion |
| DZP | diazepam |
| FD-4 | (FITC)-labeled dextrans having an |
| | average molecular weight of 4 kD |
| FD-20 | (FITC)-labeled dextrans having an |
| | average molecular weight of 20 kDa |
| FD-70 | (FITC)-labeled dextrans having an |
| | average molecular weight of 70 kDa |
| FD-250 | (FITC)-labeled dextrans having an |
| | average molecular weight of 250 kDa |
| FITC | fluorescein isothiocyanate |
| FL | fluorescein |
| fᵤ | unbound fraction |
| HSA | human serum albumin |
| MAN | mannitol |
| PBS | phosphate-buffered saline |
| PRO | propanonol |
| QND | quinidine |
| SA | salicylate |
| TRP | L-tryptophan |
| v/v | fractional volume |
| VER | verapamil |
| VPA | valproic acid, valproate |
| w/w | fractional weight |

### BACKGROUND ART

In vitro techniques for the analysis of ligand affinity and the extent of protein binding include equilibrium dialysis, ultrafiltration and ultracentrifugation. In the case of equilibrium dialysis and ultrafiltration, the protein of interest and a ligand are allowed to reach equilibrium binding in the presence of a semi-permeable membrane that permits movement of unbound ligand and restricts movement of bound ligand (Pacifici G M & Viani A, 1992). In the case of ultracentrifugation, protein-bound ligand is separated from unbound ligand by forcing the protein out of solution. However, non-specific binding of ligands to the membrane or to the apparatus can invalidate measurement of the unbound fraction. For some ligands, the extent of binding to a target protein cannot be reliably analyzed using available methods. Further, conventional membrane-based methods are labor-intensive and slow, and therefore not amenable to high throughput analysis.

Determination of unbound ligand fraction is particularly relevant to drug biodistribution. In the case of intravenous administration of a drug compound, binding of the drug to plasma proteins can substantially limit delivery of the drug to the site in need of treatment. A determination of the degree of ligand binding to plasma proteins can be used to predict the disposition of the drug in the body. See e.g., Parikh H H et al. (2000) Pharm Res 17:632-637; Trung A H et al. (1984) Biopharm Drug Dispos 5:281-290; Suarez Varela et al. (1992) J Pharm Sci 81:842-844; Ascoli G et al. (1995) J Pharm Sci 84:737-741; Barré J et al. (1985) Clin Chem 31:60-64; Mendel C M (1990) J Steroid Biochem Mol Biol 37:251-255; and Mendel C M et al. (1990) J Steroid Biochem Mol Biol 37:245-250.

Thus, there exists a long-felt need in the art for a method for rapid assessment of ligand-protein interactions that is applicable to a substantial variety of ligands. Preferably, the method can be used to determine an unbound ligand fraction for any ligand.

To meet this need, the present invention provides an apparatus and method for rapid analysis of ligand binding to a target protein. The apparatus comprises a packed-bed charcoal cartridge that is amenable to high throughput processing of samples. Using such an apparatus, a sample comprising ligand and a target protein can be evaluated to determine the percentage of unbound ligand.

### SUMMARY OF INVENTION

The present invention provides a method for evaluating binding of a ligand to a target protein, the method comprising: (a) providing a sample comprising a target protein and a ligand, wherein the target protein and ligand are suspected to be bound reversibly together in a complex; (b) preconditioning activated charcoal with the target protein; (c) contacting the sample with the pre-conditioned activated charcoal for a time sufficient to allow for adsorption of unbound ligand to the activated charcoal; (d) eluting the sample from the activated charcoal; and (e) determining an amount of ligand in the eluted sample to thereby evaluate binding of the ligand to the target protein.

The present invention further provides a method for evaluating binding of a ligand to a target protein, the method comprising: (a) providing a sample comprising a target protein and a first ligand, wherein the first ligand comprises a detectable label, and wherein the target protein and first ligand are suspected to be bound reversibly together in a complex; (b) contacting the sample with a candidate second ligand for a time sufficient for displacement of the first ligand from the complex by the second ligand; (c) preconditioning activated charcoal with the target protein; (d) contacting the sample of (b) with the pre-conditioned activated charcoal for a time sufficient to allow for adsorption of unbound first ligand to the activated charcoal; (e) eluting the sample from the activated charcoal; and (1) determining an amount of first ligand in the eluted sample to thereby evaluate binding of the second ligand to the target protein.

In a preferred embodiment of the invention, the method can further comprise employing a first ligand that comprises a ligand that binds a specific binding site of a target protein. In this case, an amount of the first ligand in the eluted sample is determined to thereby evaluate binding of the second ligand to the specific ligand binding site of a target protein.

Also provided is a method for evaluating the susceptibility of a candidate drug to binding a protein found in the circulating blood of a warm-blooded vertebrate. In a preferred embodiment, the method comprises: (a) providing a sample comprising a target protein and a ligand, wherein the ligand comprises a detectable label, and wherein the target protein and ligand are suspected to be bound reversibly together in a complex; (b) contacting the sample with a candidate drug for a time sufficient for displacement of the ligand from the complex by the candidate drug; (c) preconditioning activated charcoal with the target protein; (d) contacting the sample of (b) with the pre-conditioned activated charcoal for a time sufficient to allow for adsorption of unbound ligand to the activated charcoal; (e) eluting the sample from the activated charcoal; and (f) determining an amount of ligand in the eluted sample to thereby evaluate the susceptibility of the candidate drug to binding a protein found in the circulating blood of a warm-blooded vertebrate.

In accordance with the disclosed method, providing a sample can comprise contacting a matrix comprising a target protein with at least one ligand for a time sufficient to allow for binding of the at least one ligand by the target protein. Such contacting a matrix comprising a target protein with at least one ligand can comprise creating a suspension of the matrix comprising a target protein and the at least one ligand. A time sufficient to allow for binding will typically comprise a duration equal to or less than about 30 minutes. A preferred volume of sample to be used in performing the disclosed method comprises about 200 μl.

In a preferred embodiment of the invention, the matrix comprising a target protein is blood plasma, preferably human blood plasma. Representative plasma proteins that are important for binding interactions include but are not limited to serum albumin and α₁-acid-glycoprotein.

A ligand to be evaluated in accordance with the disclosed method can comprise a chemical compound, a peptide, an oligonucleotide, a small molecule, or combinations thereof. In a preferred embodiment, the ligand is a candidate drug. Optionally, the ligand can further comprise a detectable label.

Methods of the present invention that employ a detectably labeled first ligand to evaluate ligand binding of a candidate second ligand or drug preferably employ a first ligand that binds a plasma protein. More preferably, the first ligand comprises a ligand that binds to serum albumin or to α₁-acid-glycoprotein. Even more preferably, the first ligand comprises a ligand that binds to a specific site on serum albumin or to a specific site on α₁-acid-glycoprotein. glycoprotein.

In a more preferred embodiment, the first ligand comprises a ligand that binds a specific binding site of a target protein, preferably site I, site II, or site III of human serum albumin. The first ligand can comprise a site I-binding ligand selected from the group consisting of a cumarin and a pyrazolidine, and is more preferably selected from the group consisting of valproate, diphenylhydantoin, or salicylate. Alternatively, the first ligand can comprise a site II-binding ligand selected from the group consisting of a benzodiazepine, an arylpropionate, and L-tryptophan, more preferably the site II-binding ligand diazepam. The first ligand can also comprise the site III-binding ligand digitoxin.

The present invention also provides a method for evaluating ligand binding to a target protein that further comprises a novel preconditioning step. Preconditioning activated charcoal comprises contacting activated charcoal with the target protein or with a protein similar to the target protein for a time sufficient to allow for adsorption of the target protein or of the protein similar to the target protein to the activated charcoal. Preferably, a time sufficient for adsorption to the activated charcoal comprises about 1 second. Also preferably, the preconditioning comprises: (a) preconditioning activated charcoal immediately prior to contacting the sample with the preconditioned activated charcoal; or (b) preconditioning activated charcoal within 24 hours prior to contacting the sample with the preconditioned activated charcoal and rinsing the preconditioned activated charcoal immediately prior to contacting the sample with the preconditioned activated charcoal.

Preconditioning can also comprise: (a) applying the target protein or a protein similar to the target protein to a packed-bed activated charcoal cartridge, and (b) eluting the target protein or the protein similar to the target protein from the packed-bed activated charcoal cartridge, whereby the activated charcoal is pre-conditioned. Preferably, applying the target protein or the protein similar to the target protein comprises providing a solution having a volume and a concentration of the target protein or of the protein similar to the target protein, wherein the volume of the solution comprises a volume approximately equal to a volume of the sample, and wherein the concentration of the target protein or of the protein similar to the target protein in the solution comprises a concentration approximately equal to a concentration of the target protein in the sample.

In one embodiment of the invention, the disclosed method for evaluating ligand binding to a target protein employs dextran-coated charcoal. Preferably, the dextran-coated charcoal comprises dextrans having an average molecular weight of about 35 kDa to about 200 kDa, more preferably about 50 kDa to about 150 kDa, and even more preferably about 75 kDa to about 80 kDa.

Also preferably, the dextran-coated charcoal comprises a fractional weight of about 10% to about 80% dextran, more preferably about 10% to about 50% dextran, and still more preferably about 10% dextran. The dextran-coated charcoal also preferably comprises a mass of about 5 mg to about 100 mg, more preferably about 5 mg to about 50 mg, and still more preferably about 20 mg.

In a preferred embodiment of the invention, contacting the sample with pre-conditioned activated charcoal comprises applying the sample to a packed-bed activated charcoal cartridge. A time sufficient to allow for adsorption of unbound ligand to the activated charcoal preferably comprises about 1 second.

In another preferred embodiment of the invention, eluting the sample comprises applying suction to the sample, whereby the sample is separated from the activated charcoal.

In accordance with the present inventive method, determining an amount of ligand in the eluted sample can comprise performing mass spectrometry analysis of the eluted sample. Alternatively, determining an amount of ligand in the eluted sample can comprise detecting a detectably labeled ligand.

The present invention further provides a packed-bed charcoal cartridge for evaluating ligand binding to a target protein. The packed-bed charcoal cartridge comprises: (a) a column comprising a sample chamber, a sample addition port, and a sample elution port adapted for fluid/gaseous communication with a suction source; and (b) an activated charcoal packed-bed positioned between the sample chamber and the sample elution port, wherein the charcoal packed-bed is in fluid/gaseous communication with the sample chamber and with the sample elution port. Preferably, the activated charcoal comprises dextran-coated charcoal.

In a preferred embodiment, the column of a packed-bed charcoal cartridge comprises a sample chamber capable of holding about one (1) milliliter of liquid volume. More preferably, the column comprises a 1-ml PREPSEP® column, and a bottom of the sample chamber comprises a frit. In another preferred embodiment, the packed-bed charcoal cartridge further comprises a filter positioned adjacent to and below the frit, preferably a 1-cm glass filter.

The present invention further provides an apparatus for high-throughput analysis of ligand binding to a protein. The apparatus comprises an array of packed-bed activated charcoal cartridge units as disclosed herein. Preferably, the array comprises 96 packed-bed activated charcoal cartridge units or an integer multiple thereof (e.g. 2, 3, 4, 5, 10, 40, 100, etc.).

Also provided is a method for preparing a packed-bed activated charcoal cartridge unit. The method comprises: (a) providing a column comprising a sample chamber, a sample addition port, a sample elution port adapted for fluid/gaseous communication with a suction source, and a barrier positioned between the sample chamber and the sample elution port; (b) applying activated charcoal in a liquid suspension to the column; and (c) eluting the liquid from the column, whereby the activated charcoal is packed adjacent barrier, and whereby a packed-bed activated charcoal cartridge is prepared.

Accordingly, it is an object of the present invention to provide a method for evaluation ligand-protein interactions and a packed-bed dextran-coated charcoal matrix that can be used to perform the disclosed method. The object is achieved in whole or in part by the present invention.

An object of the invention having been stated herein above, other objects will become evident as the description proceeds when taken in connection with the accompanying Examples as best described herein below.

### BRIEF DESCRIPTION OF THE DRAWINGS

FIG. 1 is a front perspective view of a representative packed-bed activated charcoal cartridge of the present invention.

FIGS. 2A-2C are graphs depicting the adsorption of radiolabeled ligands to a packed-bed activated charcoal cartridge in the presence and absence of HSA.

FIG. 2A is a graph depicting the adsorption of [³H]-VPA to a packed-bed DCC cartridge comprising 20 mg of DCC, 10% w/w dextran at timepoints subsequent to applying the labeled ligand to the cartridge. (◯) adsorption in the presence of HSA; (●) adsorption in isotonic PBS (I-PBS) in the absence of HSA; min, minutes. Each data point represents the mean adsorption±standard error of three adsorption measurements.

FIG. 2B is a graph depicting the adsorption of [¹⁴C]-DZP to a packed-bed DCC cartridge comprising 20 mg of DCC, 10% w/w dextran at timepoints subsequent to applying the labeled ligand to the cartridge. (□) adsorption in the presence of HSA; (▪) adsorption in isotonic PBS (I-PBS) in the absence of HSA; min, minutes. Each data point represents the mean adsorption +standard error of three adsorption measurements.

FIG. 2C is a graph depicting the adsorption of [³H]-DGT to a packed-bed DCC cartridge comprising 20 mg DCC, 10% w/w dextran, at timepoints subsequent to applying the labeled ligand to the cartridge. (Δ) adsorption in the presence of HSA; (▴) adsorption in isotonic PBS (I-PBS) in the absence of HSA; min, minutes. Each data point represents the mean adsorption±standard error of three adsorption measurements.

FIGS. 3A-3C are graphs depicting the adsorption of radiolabeled ligands to an activated charcoal cartridge as a function of the unbound fraction of ligand.

FIG. 3A is a graph depicting [³H]-VPA adsorption (●) at 1 minute following application to a DCC cartridge comprising 20 mg DCC, 10% w/w dextran. [³H]-VPA was diluted in HSA solution. fᵤ, unbound fraction. Each data point represents the mean adsorption±standard error of three adsorption measurements.

FIG. 3B is a graph depicting [¹⁴C]-DZP adsorption (▴) at 1 minute following application to a DCC cartridge comprising 20 mg DCC, 10% w/w dextran. [¹⁴C]-DZP was diluted in HSA solution. fᵤ, unbound fraction. Each data point represents the mean adsorption±standard error of three adsorption measurements.

FIG. 3C is a graph depicting [³H]-DGT adsorption (▴) at 1 minute following application to a DCC cartridge comprising 20 mg DCC, 10% w/w dextran. [3H]-DGT was diluted in HSA solution. fᵤ, unbound fraction. Each data point represents the mean adsorption±standard error of three adsorption measurements.

FIGS. 4A-4C are graphs depicting adsorption of radiolabeled ligands to an activated charcoal cartridge as a function of the unbound fraction of ligand at 1 second and 2 hours following application of ligands to the cartridge.

FIG. 4A is a graph depicting [³H]-VPA adsorption at 1 second and 2 hours following application to a DCC cartridge comprising 20 mg DCC, 10% w/w dextran, [³H]-VPA was diluted in HSA solution. (●) adsorption at 1 second; (◯) adsorption at 2 hours; fᵤ, unbound fraction. Each data point represents the mean adsorption±standard error of three adsorption measurements.

FIG. 4B is a graph depicting [¹⁴C]-DZP adsorption at 1 second and 2 hours following application to a DCC cartridge comprising 20 mg DCC, 10% w/w dextran, [¹⁴C]-DZP was diluted in HSA solution. (▪) adsorption at 1 second; (□) adsorption at 2 hours; fᵤ, unbound fraction. Each data point represents the mean adsorption±standard error of three adsorption measurements.

FIG. 4C is a graph depicting [³H]-DGT adsorption at 1 second and 2 hours following application to a DCC cartridge comprising 20 mg DCC, 10% w/w dextran. [³H]-DGT was diluted in HSA solution. (♥) adsorption at 1 second; (Δ) adsorption at 2 hours; fᵤ, unbound fraction. Each data point represents the mean adsorption±standard error of three adsorption measurements.

FIGS. 5A-5B are graphs depicting adsorption of radiolabeled HSA site I-specific ligands to a DCC cartridge comprising 20 mg, 10% w/w dextran, at 1 second and 1 minute following application of ligands to the cartridge.

FIG. 5A is a graph depicting adsorption of [³H]-DPH as a function of unbound fraction. (▪) adsorption at 1 second; (□) adsorption at 1 minute; fᵤ, unbound fraction. Each data point represents the mean adsorption±standard error of three adsorption measurements.

FIG. 5B is a graph depicting [¹⁴C]-SA adsorption at timepoints following application to a DCC cartridge comprising 20 mg DCC, 10% w/w dextran. [³H]-DGT was diluted in HSA solution. (▴) adsorption at 1 second; (Δ) adsorption at 1 minute; fᵤ, unbound fraction. Each data point represents the mean adsorption±standard error of three adsorption measurements.

FIGS. 6A-6B are graphs depicting adsorption of radiolabeled ligands applied as a group to an activated charcoal cartridge as a function of unbound fraction.

FIG. 6A is a graph depicting adsorption of radiolabeled ligands at 1 second following application of ligands as a group to a DCC cartridge comprising 20 mg DCC, 10% w/w dextran (▪). The linear regression, r², was determined to be r²=0.92. DZP, diazepam; DGT, digitoxin; VPA, valproate; SA, salicylate; DPH, diphenylhydantoin; QND, quinidine; VER verapamil; PRO propanolol, TRP, tryptophan. Each data point represents the mean adsorption±standard error of three adsorption measurements.

FIG. 6B is a graph depicting adsorption of radiolabeled ligands at 1 minute following application of ligands as a group to a DCC cartridge comprising 20 mg DCC, 10% w/w dextran (●). The exponential rise to maximum, r², was determined to be r²=0.78. DZP, diazepam; DGT, digitoxin; VPA, valproate; SA, salicylate; DPH, diphenylhydantoin; QND, quinidine; VER verapamil; PRO propanolol, TRP, tryptophan. Each data point represents the mean adsorption±standard error of three adsorption measurements.

### DETAILED DESCRIPTION OF THE INVENTION

I. Definitions

While the terms used to describe the present invention are considered to be well known in the art, the following definitions are provided for convenience to facilitate understanding of the invention.

The term “about”, as used herein when referring to a value or to an amount of mass, weight, time, volume, or percentage is meant to encompass variations of ±20% or ±10%, more preferably ±5%, even more preferably ±1%, and still more preferably ±0.1% from the specified amount, as such variations are appropriate to perform the disclosed method.

The term “matrix” as used herein comprises any heterogeneous mixture, suspension or solution comprising a target protein. In a preferred embodiment, a matrix comprises blood plasma, including blood serum, from a warm-blooded vertebrate. Preferably a warm-blooded vertebrate is a mammal, and more preferably a human.

The term “target protein” comprises any endogenous protein, or portion thereof, wherein the binding characteristics of a ligand to the protein is sought. Preferably, a target protein is capable of binding to a ligand. Also preferably, a target protein comprises a plasma protein including but not limited to human serum albumin (HSA) and α₁-acid glycoprotein.

The term “similar”, as used herein to refer to describe a protein that is similar to a target protein, refers to a protein suspected of having similar ligand-binding features. A protein derived from an alternative species that is homologous to a target protein can be described as similar to the target protein. For example, bovine serum albumin is considered to be similar to human serum albumin or other plasma proteins having similar ligand-binding features.

The term “ligand” as used herein refers to any bioactive molecule, including a protein, a peptide, a nucleic acid, a lipid, a chemical compound, and combinations thereof. In one embodiment, a ligand is a candidate drug, preferably a candidate drug intended for intravenous administration to a subject.

In a preferred embodiment, a ligand to be used in accordance with the method of the present invention further comprises a detectable label. The term “detectable label” as used herein refers to a molecule is readily detected using art-recognized techniques. For example, a detectable label can comprise a radioisotope, an epitope label, a luminescent label, or a fluorescent label. Preferably, a detectable ligand does not alter the protein-binding characteristics of the ligand.

The term “binding” as used herein refers to site-specific, saturable, and reversible binding of a ligand to a protein. The term “equilibrium binding” refers to a situation wherein a rate of association of a ligand and a protein to form a complex is equal and opposite to a rate of dissociation of the complex as apo-protein and unbound ligand.

The term “time sufficient for binding” as used herein refers to a temporal duration that is sufficient for binding of a ligand to a target protein. The time sufficient for binding can be a duration sufficient to achieve greater than about 50% of equilibrium binding, more preferably greater than about 75% of equilibrium binding, even more preferably greater than about 90% of equilibrium binding, still more preferably greater than about 95% of equilibrium binding, and still more preferably greater than about 99% of equilibrium binding. In accordance with the methods disclosed herein, a time sufficient for binding will typically comprise about 15 minutes to about 60 minutes, more preferably about 30 minutes. Typically, the binding interaction is performed at 37° C.

The term “activated charcoal” as used herein refers to charcoal particles that are coated with a protein, such that the coated charcoal displays a capacity for rapid adsorption of unbound ligands.

The term “adsorption” as used herein refers to adherence of a molecule, including a protein or a ligand, to a surface. Adsorption can occur at arbitrary sites on the surface. In accordance with the preserit inventive method, a preferred surface for adsorption comprises activated charcoal, more preferably dextran-coated charcoal.

Adsorption of a molecule to charcoal can be influenced by temperature, nature of a solvent comprising the molecule, charcoal surface area, pore structure, nature of the solute, pH, the presence of inorganic salts, and the availability of competing ligands (Cooney D, 1995). Most of the factors remain consistent when using a variety of adsorbing molecules, although some differences are in the nature of the molecule itself. Although considered a neutral substance, the net charge of the activated charcoal surface is negative due to surface adsorption of OH⁻ ions. In general, the lower the aqueous solubility and the larger the molecule (in a series of compounds of similar structure), charcoal adsorption is greater. For compounds with dissimilar structure, side groups, substituent position, and molecular structure can be important for dictating the extent of adsorption. Hydroxyl, amino and sulfonic groups usually decrease adsorption while nitro groups often increase adsorption. Aromatic compounds are more adsorbable than aliphatic compounds and branched-chain molecules are more adsorbable than straight-chain molecules. Thus, for performance of the present inventive method, the above-mentioned parameters for influencing adsorption of a molecule to activated charcoal can be modified to promote a level of adsorption suitable for determining protein-ligand binding as disclosed herein.

The term “time sufficient for adsorption” as used herein refers to a temporal duration that is sufficient for adsorption of a ligand or target protein to activated charcoal. When a matrix comprising a ligand, a target protein, and complexes thereof, is applied to activated charcoal, preferably a time sufficient for adsorption does not disrupt equilibrium binding between the ligand and target protein. A time sufficient for adsorption can comprise a temporal interval to achieve adsorption of greater than about 50% available unbound ligand, more preferably greater than about 75% available unbound ligand, even more preferably greater than about 90% available unbound ligand, still more preferably greater than about 95% available unbound ligand, and still more preferably greater than about 99% available unbound ligand.

The term “eluting” as used herein refers to separation of a sample from activated charcoal, wherein a fraction of a sample is not adsorbed to the activated charcoal and is removed from proximity to or contact with the activated charcoal. Preferably, eluting the sample and collecting the sample for analysis are performed simultaneously, for example by filtration of a sample through an activated charcoal packed-bed. Preferably, filtration is facilitated by provision of a suction source for removal of the sample. Alternatively, filtration of a sample through an activated charcoal packed-bed can be facilitated by centrifugal force.

II. Design and Optimization of a Packed-Bed Dextran-Coated Charcoal Cartridge

For use in accordance with the methods of the present invention, a packed-bed charcoal cartridge should optimally display the following characteristics: (a) a maximal rate of adsorption of unbound ligand to the activated charcoal phase; (b) a minimal rate of adsorption of protein and protein-ligand complexes; (c) a rate of substrate adsorption to activated charcoal that is retarded in the presence of binding proteins; and (d) an extent of ligand adsorption to activated charcoal that is proportional to the equilibrium unbound fraction of the ligand. The packed-bed cartridge of the present invention was optimized in view of the above-mentioned criteria and thus can be used to predict unbound ligand fraction in a sample.

A packed-bed carbon cartridge of the present invention preferably comprises dextran-coated activated carbon (DCC). More preferably, the cartridge is designed to provide a stable foundation for a packed bed of DCC, to allow ease of sample addition and elution, and to be amenable to multiplex and automated formats. Optimization of a packed-bed DCC cartridge comprises examination of charcoal mass, percent dextran coating, dextran molecular weight, and preconditioning steps. For this purpose, tritiated valproate ([³H]-VPA) was used as a model ligand for assessing binding to human serum albumin (HSA) and adsorption to DCC as described in Example 1.

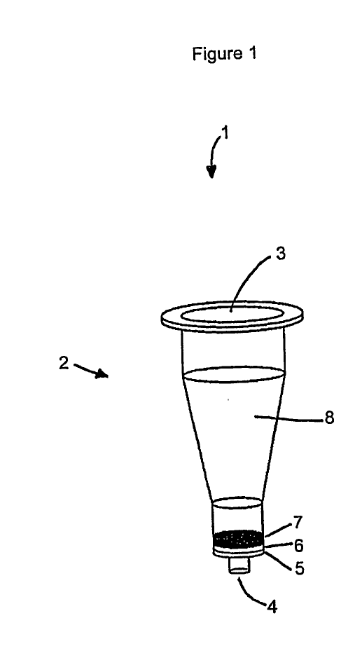

A representative packed-bed DCC cartridge 1 is shown in FIG. 1. Cartridge 1 comprises: (a) a column 2, the column having a sample addition port 3 and a sample elution port 4, wherein sample elution port 4 is adapted for fluid/gaseous communication with a suction source; and (b) a charcoal packed-bed 7.

Preferably, column 2 comprises a 1-ml PREPSEP® column (Fisher Scientific, Inc. of Pittsburgh, Pa.), wherein a bottom of sample chamber 8 comprises a frit 6 that acts as a barrier to hold the carbon in place. Also preferably, cartridge 1 further comprises a filter 5 positioned adjacent to and below frit 6, wherein filter 5 further retards flow of DCC particles. Filter 5 is preferably a 1-cm glass filter (GF/D™ binder-free glass microfiber filter available from Whatman Inc. of Clifton, N.J.).

In order to separate bound and unbound fractions of ligand, it was important to prevent significant adsorption of target protein in the DCC cartridge. Target protein adsorption was not recognized as a significant problem in a DCC suspension format (Dagenais et al., 1997). When 20 mg of DCC is added to a 1-ml suspension of 40 mg/ml HSA, the HSA mass to DCC mass ratio is 2:1 and the adsorption of HSA to charcoal is not detected. In the packed-bed format, a 200-μl volume of 40 mg/ml HSA results in a HSA mass to DCC mass ratio of 1:2.5. In the packed-bed DCC cartridge, HSA adsorption was approximately 25% between 5 and 20 mg and increased with charcoal mass over 20 mg DCC.

To circumvent this problem, the present inventive method further comprises a preconditioning step, whereby adsorption of a target protein to the DCC packed-bed is minimized during a subsequent analysis of ligand-protein binding interactions. According to the method, a volume of preconditioning matrix comprising the target protein, or a protein similar to the target protein, is applied to the DCC cartridge. The matrix is allowed to contact the DCC packed-bed for a period of time sufficient for adsorption of the target protein, and the preconditioning matrix is eluted from the column. Thus, preferably, a DCC cartridge intended for analysis of a test sample comprising a target protein is preconditioned using a matrix comprising the target protein, or a protein similar to the target protein. For example, a cartridge can be preconditioned with bovine serum albumin for the subsequent analysis of binding to human plasma proteins, including human serum albumin. Preferably, the preconditioning matrix comprises a volume and a concentration of the target protein, or a protein similar to the target protein, that is approximately equal to a volume and a concentration of the target protein in the test sample.

DCC as used in a packed-bed charcoal cartridge of the present invention can comprise variable mass and/or variable percentage of dextrans. Preferably, the average molecular weight and percentage of dextran coating is optimized to minimally adsorb a target protein. In the context of the method of the present invention, minimal adsorption of a target protein comprises adsorption of less than about 5% of a target protein.

Previous protein purification studies have reported improved reproducibility when using DCC having a relatively low average molecular weight of dextrans (Griffiths et al., 1975). However, variation of dextran molecular weight and the percent dextran coating did not alter HSA adsorption in the DCC cartridge. Therefore, a DCC packed-bed of the present invention can comprise dextran-coated charcoal comprising dextrans having an average molecular weight of about 35 kDa to about 200 kDa, more preferably about 50 kDa to about 150 kDa, and even more preferably about 75 kDa to about 80 kDa. The dextran-coated charcoal comprises a fractional weight of about 10% to about 80% dextran, more preferably bout 10% to about 50% dextran, and still more preferably about 1 0% dextran.

Thus, in a preferred embodiment of the invention, a DCC cartridge for determining an unbound ligand fraction comprises a PREPSEP® column (Fisher Scientific, Inc. of Pittsburgh, Pa.) fitted with a frit (average pore size 20 Å) and glass filter, and 20 mg of DCC in a packed-bed format. A preferred volume of matrix for use with a packed-bed DCC cartridge is about 200 μl. The volume of matrix and mass of charcoal used in the assay are suitable for a 96-well plate format and for automated performance of the method.

The present invention further provides a method for assembling a DCC cartridge for analysis of ligand-protein binding. The method comprises: (a) providing a column comprising a sample chamber, wherein a bottom of the sample chamber comprises a barrier (e.g. a frit), a filter positioned adjacent to and beneath the barrier, a sample addition port, and a sample elution port; (b) adding DCC suspending in a buffer (e.g. an aqueous solvent) to the column via the sample addition port; and (c) eluting the buffer, whereby a DCC packed-bed is formed above the barrier or frit. Preferably, the column comprises a substantially conical shape, having a relatively broad sample addition port as compared to a relatively narrow sample elution port. In a preferred embodiment of the invention, eluting the aqueous solvent comprises applying vacuum suction to the sample elution port, whereby the aqueous solvent is eluted and collected for analysis.

Referring again to FIG. 1, a method for preparing a DCC packed-bed cartridge can comprise: (a) providing a liquid suspension comprising DCC to sample chamber 8; and (b) eluting the liquid from sample chamber 8, whereby DCC is deposited as a packed-bed on frit 6, and whereby a DCC packed-bed cartridge is prepared.

III. Analysis of Ligand-Protein Interactions Using a Packed-Bed Dextran-Coated Charcoal Cartridge

The present invention also provides a method for evaluating binding of one or more ligands to a target protein. Direct assay and indirect assay formats for evaluating ligand binding are described herein below. When using a competition assay format, the invention further provides discerning ligand binding to a specific site of a target protein. Both direct assay and competition assay formats are amenable to automation and can be adapted for high throughput analysis.

Performance of the disclosed method wherein the matrix comprises multiple candidate ligands can also be used to evaluate ligand-ligand Interactions. In this case, binding of a candidate drug to a target protein can be assessed in the presence and absence of a second drug. This analysis can provide information on potential interactions between co-administered drugs.

III.A. Direct Assay

In one embodiment of the present invention, a method for evaluating ligand binding to a target protein comprises: (a) providing a sample comprising a target protein and a ligand, wherein the target protein and ligand are suspected to be bound reversibly together in a complex; (b) preconditioning activated charcoal with the target protein; (c) contacting the sample with the pre-conditioned activated charcoal for a time sufficient to allow for adsorption of unbound ligand to the activated charcoal; (d) eluting the sample from the activated charcoal; and (e) determining an amount (preferably a fractional amount) of ligand in the eluted sample to thereby evaluate binding of the ligand to the target protein. This method is referred to herein as a “direct assay format” or “non-competitive binding assay format”. The term “non-competitive binding” refers to ligand binding to a target protein, wherein binding is not influenced by provision of an exogenous second ligand that binds a same site on the target protein.

In one embodiment, determining an amount (preferably a fractional amount) of ligand can comprise detecting a detectably labeled ligand. A detectable label can comprise a radioisotope, an epitope label, a luminescent label, or a fluorescent label. Preferably, a detectable ligand does not alter the protein-binding characteristics of the ligand.

Methods for detectably labeling a ligand will vary depending on the molecular nature of the ligand. A typical method for detectably labeling a chemical compound is radiolabeling and can be accomplished using art-recognized techniques. Representative methods for protein labeling include but are not limited to radiolabeling, addition of biotin or other epitope label by cross-linking or metabolic addition (Parrott M B & Barry M A, 2000; Parrott M B & Barry M A, 2001); and fluorescent labeling (Gruber H J et al., 2000). Techniques for labeling nucleic acid ligands include but are not limited to incorporation of labeled nucleotide analogues during nucleic acid replication, transcription, or amplification; addition of an end-label during a terminal transferase reaction; and formation of triplex structures. See e.g. McPherson M et al. (eds.) (1995) PCR 2: A Practical Arproach. IRL Press, New York; Sambrook J & Russell D (2001) Molecular Cloning: A Laboratory Manual. 3rd ed. Cold Spring Harbor Laboratory Press, Cold Spring Harbor, N.Y.; and Ausubel F (ed.) (1995) Short Protocols in Molecular Biology. 3rd ed. Wiley, N.Y.

Methods for detecting a labeled ligand are selected as appropriate for a type of label employed. For example, a radio-isotopic label can be detected using liquid scintillation spectroscopy. A fluorescent label can be detected directly using emission and absorbance spectra that are appropriate for the particular label used. Fluorescent tags also include sulfonated cyanine dyes that can be detected using infared imaging.

Alternatively, an amount (preferably a fractional amount) of an unlabeled ligand can be determined using any one of a variety of methods for protein analysis, including high performance liquid chromatography (HPLC), and capillary electrophoresis. See Wahler D & Reymond J L (2001) Curr Opin Chem Biol 5:152-158; Maurer H H (2000) Comb Chem High Throughput Screen 3:467-480; and references cited therein.

The term “mass spectrometry” as used herein refers to techniques including but not limited to gas chromatography-mass spectrometry (GC-MS), liquid chromatography-mass spectrometry (LC-MS), laser-desorption mass spectrometry (LD-MS), matrix-assisted laser desorption/ionization mass spectrometry (MALDI-MS), time-of-flight mass spectrometry (TOF-MS), electrospray ionization mass spectrometry (ESI-MS); tandem mass spectroscopy, field release mass spectrometry, and combinations thereof. See e.g., Maurer H H (2000) Comb Chem High Throughput Screen 3:467-480; Karas M et al. (2000) Fresenius J Anal Chem 366:669-676; Kowalski P & Stoerker J (2000) Pharmacogenomics 1:359-366; Griffiths W J et al. (2001) Biochem J 355:545-561; U.S. Pat. Nos. 6,107,623; 6,104,028; 6,093,300; 6,057,543; 6,017,693; 6,002,127; 5,118,937; 5,952,654; and references cited therein. Such techniques are known to one of skill in the art and representative protocols for sample preparation can be found for example, in Gilar M et al. (2001) J Chromatogr A 909:111-135, U.S. Pat. No. 5,545,895, and references cited therein.

To facilitate analysis of multiple ligands, a multiplexing approach can be used similarly to that described as “cassette-accelerated rapid rat screen” (Korfmacher W A et al., 2001). Briefly, duplicate samples are prepared for analysis of a single ligand to a single target protein. Following analysis, samples are pooled such that each pooled sample comprises about 6 individual samples, or other desired number of samples. Mass spectrometry is streamlined by analyzing the samples as cassettes of six, or other desired number of samples.

For simultaneous analysis of binding of multiple candidate ligands to a single target protein, the providing of a sample can comprise contacting a target protein with a plurality of candidate ligands for a time sufficient to allow for binding of the target protein to one or more of the plurality of candidate ligands. When evaluating ligand binding in a sample comprising a target protein and a plurality of ligands, an amount (preferably a fractional amount) of each ligand can be determined in the eluted matrix by using liquid chromatography coupled to tandem mass spectroscopy (Berman J et al., 1997; McLoughlin D A et al., 1997; Olah T V et al., 1997; Beaudry F et al., 1998; Frick L et al., 1998), fast-atom bombardment mass spectrometry (Newton R P et al., 1997; Walton T J et al., 1998; White R & Manitpisitkul P, 2001), or high performance liquid chromatography (U.S. Pat. No. 5,993,662). Preferably, a plurality of candidate ligands in a sample comprises less than or equal to about 10 candidate ligands.

III.B. Analysis of Ligand-HSA Interactions Using a Direct Assay

The present invention provides a method and apparatus for evaluating ligand binding that performs well for a variety of candidate ligands. As described in Example 2, DCC adsorption of three HSA site-specific ligands, valproate (site I), diazepam (site II), and digitoxin (site III), was studied over time in the absence and presence of protein. In addition, the effect of unbound fraction on the extent of charcoal adsorption was assessed. Two alternative site I-specific markers, diphenylhydantoin (DPH) and salicylate (SA), also were examined. Finally, the extent of DCC adsorption of a set of compounds with a range of unbound fraction between 0.02 and 0.5 was evaluated in the DCC cartridge.

The HSA ligands selected for analysis have disparate molecular features. Diazepam, a cation (pKₐ=3.4) is unionized at physiologic pH and has two aromatic centers that could contribute to its highly adsorptive nature. Digitoxin is a neutral compound; however, its molecular size is greater than that of diazepam or valproate. Valproate, an anion (pKₐ=4.6) is ionized at physiologic pH and potentially repelled from adsorption at the charcoal surface.

Despite differences in ligand structure and chemistry, [³H]-VPA, [¹⁴C]-DZP and [³H]-DIGT adsorption to DCC in the absence of HSA was rapid and nearly complete. In the presence of HSA, the rate and extent of [³H]-VPA, [¹⁴C]-DZP and [³H]-DIGT adsorption was restricted. The extent of charcoal adsorption increased as a function of unbound fraction for all ligands studied. After 1 minute of exposure, the relationship between the extent of adsorption and unbound fraction could be described with an exponential rise to a maximum. When multiple ligands were assessed, a similar relation was obtained.

In order for the packed-bed DCC cartridge to be used as an in vitro method for measuring the extent of protein binding, there must be a clear relationship between the extent of charcoal adsorption and unbound fraction. For diazepam and digitoxin after 1 minute DCC exposure, the DCC was unable to discern changes in unbound fraction over 20%. Valproate adsorption was affected more by unbound fraction, which could be due to differences in protein binding and/or charcoal adsorption. A similar curve was seen when a set of multiple ligands were exposed to 1 minute DCC at physiologic HSA concentrations. Again, the extent of adsorption ceased to be affected by unbound fraction over 20%. The lack of discrimination under these conditions is due to the fact that the 1-minute exposure was sufficiently long to allow perturbation of the equilibrium between bound and unbound ligand.

The most useful relationship was obtained when the set of ligands was allowed a 1-second exposure to DCC. In this case, the correlation (r²=0.92) between the extent of adsorption and unbound fraction was linear, such that the extent of DCC adsorption could be used to predict the unbound fraction. However, the cartridge was unable to discern unbound fraction over 50% unbound (i.e., ranges of protein binding that are generally considered to be insubstantial). The predictive capacity derived from this experimental design was an improvement over the 1-minute exposure time. Data corrected for the adsorption of ligand in isotonic phosphate buffered saline in the absence of HSA provided no improvement in the correlation between the extent of adsorption and unbound fraction. Thus, the present inventive method is particularly useful for predicting unbound fraction when a ligand is predominantly bound to its target protein.

III.C. Competition Assay

In another embodiment of the present invention, a method for evaluating ligand binding to a target protein comprises: (a) providing a sample comprising a target protein and a first ligand, wherein the first ligand comprises a detectable label, and wherein the target protein and first ligand are suspected to be bound reversibly together in a complex; (b) contacting the sample with a candidate second ligand for a time sufficient for displacement of the first ligand from the complex by the second ligand; (c) preconditioning activated charcoal with the target protein; (d) contacting the sample of (b) with the pre-conditioned activated charcoal for a time sufficient to allow for adsorption of unbound first ligand to the activated charcoal; (e) eluting the sample from the activated charcoal; and (f) determining an amount (preferably a fractional amount) of first ligand in the eluted sample to thereby evaluate binding of the second ligand to the target protein.

Thus, the present invention further provides a method for evaluating ligand-protein binding that is based on competitive binding between a first ligand and a second ligand. The term “competitive binding” as used herein refers to direct displacement of a first ligand from a binding site on a target protein by a second ligand that specifically binds the same site. A candidate second ligand can be identified as binding a target protein at a particular site by observing displacement of a detectably labeled first ligand known to bind that same site.

In one embodiment, a target protein comprises multiple binding sites that show different ligand specificity. In this case, a site-specific ligand can be used as the detectably labeled first ligand. In this case, the method of the present invention can be used to distinguish binding of one or more ligands to a specific site on a target protein.

The term “specific site” as used herein refers to a ligand-binding site on a target protein comprising a space or surface defined by a subset of target protein amino acids. Alternatively, the term “specific binding” can refer to a binding site on a target protein that shows selective binding. The term “selective binding”, as used herein to describe binding to a specific site, refers to binding of a subset of ligands for a target protein.

At least six classes of primary (high-specificity) binding sites have been identified on HSA, and a larger number of secondary (lower specificity) binding sites. The warfarin site (site I) primarily interacts with cumarines, salicylates, and pyrazolidines, and the indole site (site II) specifically binds benzodiazepines, arylpropionates, and L-tryptophan. Site III can be specifically bound by digitoxin. Thus, a detectably labeled site-specific HSA ligand can be used in accordance with the method of the present invention to evaluate binding of a candidate drug to a particular binding site on HSA.

For simultaneous analysis of numerous candidate ligands to a same target protein, a competitive assay format as described herein above is preferred to avoid the need to label each candidate ligand. Further, this method can be automated and adapted for high-throughput analysis. For example, labeled valproate could be employed as a first HSA ligand in performance of the disclosed method to screen a library of compounds for binding to HSA site I. According to the disclosed method, an amount of eluted valproate can be used to determine an amount of ligand binding to HSA site I.

IV. Applications

The method of the present invention can be used to evaluate binding of any ligand to any target protein. The disclosed method is also useful for evaluating ligand binding to a specific site on a target protein.

In a preferred embodiment of the invention, the method is used to determine unbound fraction of a candidate drug in the presence of plasma proteins. Performance of the method can be included in a drug development program for predicting drug disposition and activity. See Huang J D & Oie S (1982) J Pharmacol Exp Ther 223:469-471 and Qin M et al. (1994) J Pharmacol Exp Ther 269:1176-1181. The dextran-coated charcoal cartridge as disclosed herein is amenable to rapid analysis and high-throughput formats and thus is applicable to the current accelerated pace of drug discovery. The present invention thus further provides an apparatus for high-throughput analysis of ligand binding to a protein. The apparatus comprises an array of packed-bed activated charcoal cartridge units as disclosed herein. Preferably, the array comprises 96 packed-bed activated charcoal cartridge units or an integer multiple thereof (e.g. 2, 3, 4, 5, 10, 40, 100, etc.).

### EXAMPLES

The following Examples have been Included to illustrate modes of the invention. Certain aspects of the following Examples are described in terms of techniques and procedures found or contemplated by the present inventor to work well in the practice of the invention. The Examples illustrate standard laboratory practices of the present inventor. In light of the present disclosure and the general level of skill in the art, those of skill can appreciate that the following Examples are intended to be exemplary only and that numerous changes, modifications, and alterations can be employed without departing from the scope of the invention.

### Example 1

### Design and Optimization of a Dextran-Coated Charcoal Cartridae Summary

This Example demonstrates development of a novel packed-bed dextran-coated charcoal (DCC)-adsorption cartridge for examining the kinetics of ligand binding to target proteins in vitro. Tritiated valproate ([³H]-VPA) was used as a model ligand for assessing binding to human serum albumin (HSA) and adsorption to DCC. Optimization of the packed-bed cartridge included examination of charcoal mass, percent dextran coating, dextran molecular weight, and preconditioning steps in order to minimize HSA adsorption. The surface area available for adsorption on the DCC was characterized with various molecular weight dextrans. The kinetics of [3H]-VPA adsorption to DCC was assessed in the absence and presence of HSA. Inhibition of [³H]-VPA adsorption to DCC by HSA was compared with the unbound fraction of [³H]-VPA, as determined by ultrafiltration.

The optimized system utilized 20 mg of DCC (77 kDa; 10% w/w dextran) in a packed-bed format pre-conditioned immediately prior to use with HSA (40 mg/ml; 200 μl). The available surface area on DCC decreased as dextran molecular weight increased (4-250 kDa). In the absence of HSA, [³H]-VPA adsorption to DCC was rapid and nearly complete, while in the presence of HSA, [3H]-VPA adsorption was limited to 70% at 5 minutes. As the unbound fraction of [³H]-VPA increased, adsorption to DCC increased, demonstrating an extent of adsorption as a useful indicator of unbound fraction.

### Materials and Methods

Materials. Properties of the activated carbon (DARCO® G-60, 100 mesh, powder available from Aldrich of Milwaukee, Wis.) used to prepare a DCC cartridge of the present invention are summarized in Table 1 below (Adapted from ( - - -, 1965; Cheremisinoff P & Morresi A, 1978). Dextrans (average molecular weight: 35-45 kDa, 65-85 kDa and 100-200 kDa), fluorescein, fluorescein isothiocyanate(FITC)-labeled dextrans (average molecular weight: 4.4, 19.5, 77 and 282 kDa) and human serum albumin (HSA, Fraction V powder) were purchased from Sigma of St. Louis, Mo.). Empty PREPSEP® columns and 1-cm glass filters (WHATMAN® GF/D™ binder-free glass microfiber filters) were purchased from Fisher Scientific of Pittsburgh, Pa. Tritiated mannitol ([³H]-MAN) was purchased from Sigma of St. Louis, Mo. Tritiated valproate ([³H]-VPA) was purchased from Amersham Pharmacia Biotech Inc. of Piscataway, N.J.). Radiochemical purity of [³H]-VPA was determined to be >98% by thin layer chromatography (mobile phase: toluene:methanol:acetic acid [45:8:4]; stationary phase: WHATMAN® 250-μm silica plate).

TABLE 1

| | | |
|---|---|---|
| ORIGIN: | Lignite | |
| SURFACE AREA: | 750-800 m²/g | |
| BULK DENSITY: | 25 lb/ft³ | |
| STORAGE SPACE: | 100 ft³/ton | |
| PARTICLE SIZE: | <100 mesh | 95% |
| | <325 mesh | 70% |
| PORE VOLUME: | 1 ml/g | |
| PORE VOLUME DISTRIBUTION: | <20 Å | 10% |
| | 20-50 | 10% |
| | 50-100 | 30% |
| | 100-500 | 35% |
| | >500 Å | 15% |
| MEAN PORE RADIUS: | 25 Å | |

Dextran-Coated Charcoal Procedure. Activated charcoal (2 g) was washed five times to remove small suspended charcoal particles. Each wash involved rinsing the activated charcoal in deionized water followed by centrifugation (500×g; 2 minutes). Additional washes comprised allowing charcoal to settle by gravity (10 minutes) twice in deionized water and removing the supernatant. The final activated charcoal slurry was transferred to a 500-ml beaker containing dextran (200-1600 mg) in approximately 300 ml of deionized water. The dextran-charcoal slurry was allowed to stir for 24 hours at room temperature to promote dextran adsorption to the charcoal particles. The resulting slurry was transferred to 50-ml conical tubes and the volume reduced by centrifugation (500×g; 2 minutes). A final wash in deionized water was performed, the dextran-coated charcoal slurry was centrifuged (500×g, 2 minutes), and the supernatant was removed. The final dextran-coated charcoal slurry was transferred to an amber glass jar and the contents shell frozen. The DCC was lyophilized at −50° C. for 48 hours to remove residual water from the preparation. DCC was stored in amber glass jars at room temperature no longer than one month prior to use.

DCC Cartridge Design. The DCC cartridge was designed to provide a stable foundation for a packed bed of charcoal and to allow ease of sample addition and elution. A preferred packed-bed DCC cartridge design is shown in FIG. 1, as disclosed herein above. Briefly, the cartridge comprised an empty PREPSEP® column commonly used for solid-phase extraction (Fisher Scientific of Pittsburgh, Pa.). A frit with an average pore size of 20 Å was provided with each column. The wide mouth of the column aided in sample addition. A glass filter (1 cm) was placed below the frit to prevent charcoal particles from eluting with the sample under vacuum. Cartridges were prepared by adding DCC in suspension (100 mg/ml in isotonic phosphate buffered saline (0.067M H₂PO₄, 0.4% (w/v) NaCl, pH 7.4) to the column and eluting the residual buffer under vacuum on a solid-phase extraction manifold (Baker-10 SPE System, available from J. T. Baker Chemical Co. of Phillipsburg, N.J.).

Parameters Evaluated for DCC Cartridge Design. The design of the packed-bed DCC cartridge was optimized in order to minimize human serum albumin (HSA) adsorption. Factors evaluated included charcoal mass (5, 10, 20, 50, 100 mg DCC), percent dextran coating (10, 20, 40, 80% w/w dextran), dextran molecular weight (average molecular weight: 35-45, 65-85 and 100-200 kDa), and preconditioning steps (isotonic PBS, dextran and HSA rinse). DCC cartridges (20 mg, 10% DCC) were prepared and evaluated for fluid recovery on the day of preparation and 24 hours after preparation. A load volume of 200 μl was selected for evaluation. Experimental factors were evaluated by adding 200 μl HSA solution (approximately 40 mg/ml) to the cartridge and eluting under vacuum after a 1-minute exposure to the DCC cartridge. Samples were collected and analyzed for protein content after elution. Data are presented as the percent of HSA recovered in the elution medium relative to the original protein concentration.

Protein Assay. Eluted samples were collected, diluted and analyzed for protein content by a modified method of Lowry O et al. (1951) with a kit available from Bio-Rad Laboratories of Hercules, California. Spectrophotometric absorbance of samples and standards was measured at 750 nm. Protein concentrations in samples were determined against bovine serum albumin (BSA) as a standard. Standard curves were generated daily with BSA in isotonic PBS.

Adsorption Surface Area Characterization. Fluorescein (FL) and tritiated mannitol ([³H]-MAN) were used as small molecule markers for the characterization of the available surface area of the DCC preparation. The effect of molecular weight on the available surface of DCC was evaluated using fluorescein isothiocyanate (FITC)-labeled dextrans of increasing molecular weight (average: 4.4 [FD-4], 19.5 [FD-20), 77 [FD-70] and 282 [FD-250] kDa). Aliquots (1 ml) of various concentrations (1-100 μM) of each marker were incubated with 5 mg of the DCC preparation at room temperature for 30 minutes. Suspensions were centrifuged at 9000×g for 10 minutes to pellet the DCC. The supernatant was analyzed for marker recovery. The surface area was evaluated further with each marker in the DCC cartridge. Each marker (200 [2l of 100 μM solution) was loaded onto the DCC cartridge. Elution was initiated and samples collected under vacuum at 1 second and at 60 seconds. Counts per minute (CPM) of original solutions, supernatant and eluted samples were determined by liquid scintillation spectroscopy of 50-μl aliquots for [³H]-MAN. FL and FITC-dextran samples were analyzed by fluorescence spectroscopy (λ_{EX}=505 nm, λ_{EM}=523 nm) with a PerkinElmer fluorimeter Model No. LS50B (PerkinElmer, Inc. of Wellesley, Mass.) and quantitated using standard curves generated for each marker.

Model Ligand Adsorption Experiments in the DCC Cartridge. Tritiated valproate ([³H]-VPA) was selected as a model ligand for evaluating xenobiotic protein binding in the packed-bed DCC cartridge. Valproate is highly protein bound (unbound fraction: 0.1) to HSA (Zaccara G et al., 1988). Charcoal adsorption experiments were conducted in the packed-bed DCC cartridge (20 mg, 10% DCC). Cartridges were prepared and preconditioned with a 40-mg/ml solution of HSA. Adsorption profiles of [³H]-VPA in the absence and presence of HSA were generated. Solutions of [3H]-VPA (10 mg/ml; 0.1 μCi/ml) in isotonic PBS and HSA solution (40 mg/ml HSA in isotonic PBS) were prepared and allowed to incubate at room temperature for 30 minutes. 200 μl samples were loaded onto the DCC cartridge. Elution was initiated and samples collected under vacuum at 1, 2, 5, 10, 20, 40, 60, 120 and 300 seconds.

Extent of charcoal adsorption of [³H]-VPA was evaluated as a function of unbound fraction. Solutions of [³H]-VPA (10 mg/ml; 0.1 μCi/ml) in isotonic PBS and dilutions of a 40 mg/ml HSA solution (100%, 10%, 1%, 0.75% and 0.5% v/v) were prepared. Solutions were allowed to incubate at room temperature for 30 minutes and 200 μl samples were loaded onto the DCC cartridge. Elution was initiated and samples collected under vacuum at 5, 10 and 60 seconds. [³H]-VPA in 50-μl aliquots of the original solutions and eluted samples was determined by liquid scintillation spectroscopy.

Ultrafiltration Experiments. Equilibrium unbound fractions of [³H]-VPA in corresponding HSA-Buffer dilutions were measured by ultrafiltration with the CENTRIFREE® centrifugal filtration device (Millipore of Bedford, Mass.). Samples (0.5 ml) were centrifuged (1500×g; 3 minutes) at 25° C. [³H]-VPA in 50-μl aliquots of the original solutions and filtrate samples was determined by liquid scintillation spectroscopy.

Statistical Analysis. Statistical analysis was performed using SAS™ v.6.12 (SAS Institute Inc. of Cary, N.C.). In all cases, p<0.05 was considered statistically significant.

### Results

Design and Optimization of the DCC Cartridge. After constructing and testing several designs consisting of a 1-cc syringe plugged with glass wool, the final cartridge comprised a PREPSEP® column (Fisher Scientific of Pittsburgh, Pa.) fitted with frit (average pore size, 20 Å) and glass filter (WHATMAN® 1-cm GF/D binder-free glass microfiber filter available from Fisher Scientific of Pittsburgh, Pa.). The DCC was applied to the frit-filter support as a suspension in isotonic PBS, and the packed-bed was formed through elution of residual isotonic PBS under vacuum. This design was simple to assemble and straightforward to use.

Adsorption of HSA to DCC increased significantly as charcoal mass increased (20-100 mg). Charcoal mass had no effect on HSA adsorption in the range of 5-20 mg. In each case, recovery was consistently incomplete (approximately 75%). In order to use the DCC cartridge system for the study of unbound fraction or dissociation of ligand from protein, the extent of HSA adsorption should preferably be no more than 5%. HSA showed a similar kinetics of adsorption to dextran-coated charcoal and non-dextran-coated charcoal in a packed-bed cartridge.

Percent dextran coating in the range of 10% to 80% and dextran average molecular weight in the range of 44 kDa to 188 kDa) had no significant effect on the recovery of HSA in elution samples. Changing these variables did not improve HSA recovery over the original cartridge conformation. No significant loss of HSA to the frit or filter was noted.

Preconditioning with either isotonic PBS (200 μl ) or dextran (200 μl of 10 mg/ml dextran) did not result in significant improvement in protein recovery. In both cases, HSA recovery was observed at about 60%. A rinse with HSA solution (200 μl of 40 mg/ml HSA) resulted in approximately 95% HSA recovery. A rinse with lesser volumes of HSA (50 μl and 100 μl of 40 mg/ml HSA) resulted in somewhat less (about 90%) and statistically less (about 80%) HSA recovery, respectively. Preconditioning with an equal volume of HSA solution therefore was incorporated prior to all subsequent experiments.

Fluid recovery was assessed in the final DCC cartridge system, and the results are summarized in Table 2. Elution volume increased linearly (slope: 0.97-0.99) with increasing load volume (50-500 μl). When cartridges were prepared 24 hours in advance, rinsed with HSA and allowed to dry (Dry), the fluid recovery volume was consistently lower (intercept: −53.2) than dry cartridges preconditioned with isotonic PBS (Pre-rinsed) or those prepared on the day of the experiment (Wet). Approximately 50 μl of the load volume was lost on dry cartridges. Pre-Rinsed and Wet cartridges revealed approximately 100% fluid recovery regardless of load volume. DCC cartridges were prepared either on the day of the experiment or 24 hours in advance and pre-rinsed for all subsequent experiments.

TABLE 2

| | SLOPE | INTERCEPT | R² |
|---|---|---|---|
| Dry | 1.04 ± 0.007 | −54.3 ± 2.9^{b} | 0.998 |
| Pre-Rinsed | 1.02 ± 0.032 | 1.2 ± 4.1 | 0.999 |
| Wet | 1.02 ± 0.011 | −1.6 ± 1.5 | 0.999 |

^{a}Data are presented as mean ± SD.
^{b}Statistically different mean from other two groups.

Characterization of DCC Available Surface. The adsorption characteristics of the small molecules studied in a DCC suspension were unequal. Fluorescein was adsorbed completely within the concentration range studied while [3H]-mannitol was adsorbed to a significantly lesser extent (approximately 25%). As molecular weight and concentration of FITC-labeled dextrans (FD) increased, the extent of DCC adsorption decreased. When partial adsorption isotherms were constructed for each marker, large molecular weight compounds (FD-70 and FD-250) were observed to achieve surface saturation at relatively low concentrations. Adsorption of FD-20 and [³H]-mannitol revealed similar isotherms approaching surface saturation of DCC at concentrations outside the concentrations studied (greater than 100 μM). FD-40 and fluorescein adsorption isotherms did not appear to approach saturation of DCC within the concentration range examined. When the data for fluorescein and FD were evaluated as the unadsorbed mass in the supernatant as a function of marker concentration and molecular weight, a relationship among DCC surface available for adsorption, compound size, and compound concentration was established.

In the DCC cartridge, the extent of marker adsorption decreased with increasing molecular weight up to FD-70 but increased with time of charcoal exposure. After immediate exposure to DCC, the amount of adsorption decreased linearly with the log of molecular weight. The apparent extent of FD-250 adsorption was greater than FD-70 adsorption; however, the apparent increase in available surface may have resulted from the torturosity of the packed-bed format. [3H]-mannitol adsorption was similar to FD-4 adsorption in the DCC cartridge.

Time Course of [³H]-VPA Adsorption in the DCC Cartridge in the Absence and Presence of Human Serum Albumin. [³H]-VPA adsorption to DCC in the absence of HSA was rapid and nearly complete (approximately 99%) after 1 minute. In the presence of HSA (40 mg/ml), valproate adsorption was restricted and reached approximately 75% of the total [³H]-VPA available in the system for adsorption when the experiment was terminated.

Extent of [³H]-VPA Adsorption as a Function of the Unbound Fraction in the DCC Cartridge. Fractional [³H]-VPA adsorption to DCC increased linearly with unbound fraction (0.1 to 0.5 unbound fraction) after 5, 10 and 60 seconds of charcoal exposure. The extent of DCC adsorption was higher after 1 minute of exposure than for 5 seconds or 10 seconds of exposure. As unbound fraction increased beyond the linear range, adsorption was limited to approximately 80%.

### Example 2

### Ligand Adsorption and HSA Binding in a Dextran-Coated Charcoal Cartridge Summary

DCC adsorption of HSA site-specific ligands was studied in the absence and presence of protein using an activated charcoal cartridge as described in Example 1. Ligands included site I-specific ligands valproate (VPA), diphenylhydantoin (DPH) and salicylate (SA); a site II-specific ligand diazepam (DZP); and a site III-specific ligand digitoxin (DIGT). [³H]-VPA, [¹⁴C]-DZP and [³H]-DIGT adsorption to DCC in the absence of HSA was rapid and nearly complete. In the presence of HSA, the rate and extent of adsorption was restricted. The extent of charcoal adsorption increased as a function of unbound fraction for all ligands studied. After a 1-minute exposure, the relationship between the extent of adsorption and unbound fraction could be described with an exponential rise to a maximum. When multiple ligands were assessed, a similar relation was obtained. When ligands were allowed a 1-second exposure to DCC, the correlation (r²=0.92) between the extent of adsorption and unbound fraction was linear, enabling a prediction of unbound fraction.

### Materials and Methods

Ligand Adsorption Experiments in the DCC Cartridge. Tritiated valproate ([³H]-VPA), ¹⁴C-labeled diazepam ([¹⁴C]-DZP), and tritiated digitoxin ([³H]-DIGT) were selected as HSA site-specific ligands for evaluating xenobiotic protein binding in the packed-bed DCC cartridge. Charcoal adsorption experiments were conducted in a packed-bed DCC cartridge (20 mg DCC, 10% w/w). Cartridges were prepared and preconditioned with a 40 mg/ml solution of HSA. Adsorption profiles of [³H]-VPA, [¹⁴C]-DZP and [³H]-DIGT in the absence and presence of HSA were generated over 5 minutes. Solutions of [³H]-VPA (10 mg/ml; 0.1 μCi/ml), [¹⁴C]-DZP (350 ng/ml; 0.1 μCi/ml), and [³H]-DIGT (25 ng/ml; 0.1 μCi/ml) in isotonic PBS and HSA solution (40 mg/ml HSA in isotonic PBS) were prepared and allowed to incubate at room temperature for 30 minutes. 200 μl samples were loaded onto the DCC cartridge. Elution was initiated and samples collected under vacuum at 1, 2, 5, 10, 20, 40, 60, 120 and 300 seconds.

Extent of charcoal adsorption of [³H]-VPA, [¹⁴C]-DZP and [3H]-DIGT was evaluated as a function of unbound fraction. Solutions of [³H]-VPA (10 mg/ml; 0.1 μCi/ml), [¹⁴C]-DZP (350 ng/ml; 0.1 μCi/ml), and [³H]-DIGT (25 ng/ml; 0.1 μCi/ml) in isotonic PBS and dilutions of a 40 mg/ml HSA solution were prepared. Solutions were allowed to incubate at room temperature for 30 minutes and 200 μl samples were loaded onto the DCC cartridge. Elution was initiated and samples were collected under vacuum at 1 second and at 60 seconds. Adsorption of each ligand also was evaluated after 2 hours of DCC exposure. Long-term exposure was conducted in the DCC suspension format (Dagenais et al., 1997). [³H]-VPA, [¹⁴C]-DZP and [³H]-DIGT in 50-μl aliquots of the original solutions and eluted samples were determined by liquid scintillation spectroscopy.

Ultrafiltration Experiments. Equilibrium unbound fractions of [3H]-VPA, [¹⁴C]-DZP and [³H]-DIGT in corresponding HSA-Buffer dilutions were measured by ultrafiltration with a CENTRIFREE® centrifugal filtration device (Millipore of Bedford, Mass.). Samples (0.5 ml) were centrifuged (1500×g; 3 minutes) at 25° C. [³H]-VPA, [¹⁴C]-DZP and [³H]-DIGT in 50-μl aliquots of the original solutions and filtrate samples were determined by liquid scintillation spectroscopy.

Evaluation of Alternative HSA Site I-Specific Ligands. Alternative HSA site I-specific ligands included tritiated diphenylhydantoin ([³H]-DPH, available from New England Nuclear Life Sciences Products, Inc. of Boston, Mass.) and ¹⁴C-labeled salicylate ([¹⁴C]-SA, available from American Radiolabeled Chemicals, Inc. of St. Louis, Mo.) were evaluated for the extent of adsorption and unbound fraction after 1 second and 1 minute of DCC exposure. DCC adsorption and ultrafiltration experiments were conducted as described in Example 1.

Multiple Ligand Adsorptions in the DCC Cartridge. The extent of DCC adsorption of a set of compounds with a range of unbound fraction between 0.02 and 0.5 was evaluated in the DCC cartridge after 1 second and 1 minute of exposure in HSA-Buffer. The compounds tested are listed in Table 3 (a, available from American Radiolabeled Chemicals, Inc. of St. Louis, Mo.; b, available from Amersham Pharmacia Biotech Inc. of Piscataway, N.J.; c, available from New England Nuclear Life Sciences Products, Inc. of Boston, Mass.).

TABLE 3

| | MOLECULAR | | | UNBOUND | |
|---|---|---|---|---|---|
| LIGAND | WEIGHT | CONCENTRATION | | FRACTION | REFERENCE |
| [³H]-VPA^{a} | 144.21 | 10 | μg/ml | 0.1 | (Zaccara G et al., 1988) |
| [¹⁴C]-DZP^{b} | 284.74 | 350 | ng/ml | 0.02 | (Greenblatt DJ et al., 1980) |
| [³H]-DIGT^{c} | 764.95 | 25 | ng/ml | 0.05 | (Mooradian AD, 1988) |
| [³H]-DPH^{c} | 252.27 | 15 | μg/ml | 0.12 | (Grasela TH et al., 1983) |
| [¹⁴C]-SA^{a} | 138.12 | 10 | μg/ml | 0.05 | (Furst DE et al., 1979) |
| [³H]-PRO^{b} | 259.35 | 20 | ng/ml | 0.13 | (Riddell JG et al., 1987) |
| [³H]-QND^{a} | 324.42 | 2 | μg/ml | 0.13 | (Ochs HR et al., 1980) |
| [³H]-TRP^{b} | 204.23 | 500 | ng/ml | 0.5 | (Mingrone G et al., 1997) |
| [³H]-VER^{c} | 454.61 | 120 | ng/ml | 0.1 | (Echizen H & Eichelbaum M, 1986) |

Adsorption of the ligands in isotonic PBS also was determined after 1 second of exposure to DCC. Adsorption and ultrafiltration experiments were conducted as described in Example 1.

Data Analysis. Data analysis, curve fitting and linear regression were performed using SIGMA PLOT™ 2000 v.6.0 (SPSS Inc. of Chicago, Ill.). Curve fitting for non-linear regression included analysis of exponential rise to maximum and capacity-limited isotherm equations. Fitting of the two equations either excluded or included an intercept term. Equations were fit to the ligand sets simultaneously due to sparse or incomplete data sets and parameter estimates and standard errors represent the error in the parameter estimate, not the error in individual fits. The equation judged to be appropriate was chosen based on residual error and visual examination of goodness-of-fit.

### Results

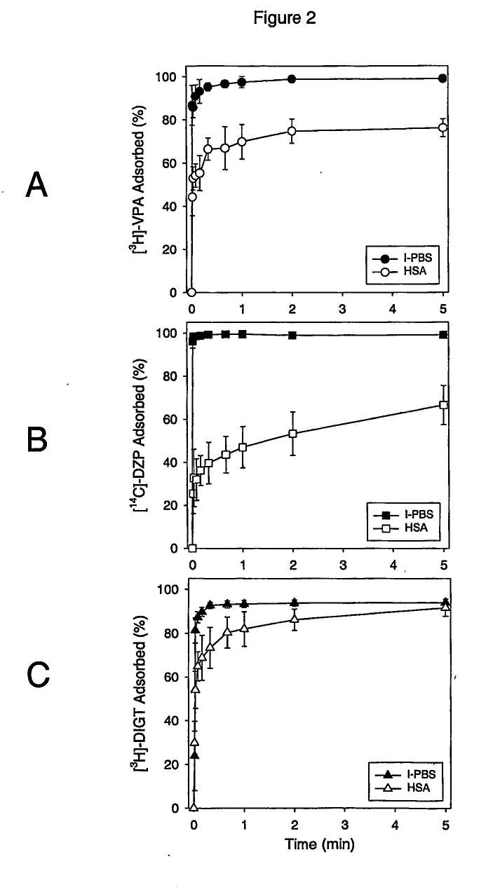

Time Course of Ligand Adsorption in the Presence and Absence of HSA. FIGS. 2A-2C present graphs showing the percentage of [³H]-VPA, [¹⁴C]-DZP and [³H]-DIGT adsorption to DCC in the absence of HSA was rapid and nearly complete (approximately 99%) after 1 minute. Although results were similar for each of the ligands, it is noteworthy that the rate of adsorption in the absence of HSA appeared faster for [¹⁴C]-DZP than for [³H]-VPA or [³H]-DIGT. In the presence of HSA (40 mg/ml), valproate adsorption was restricted and reached approximately 75% of the total [³H]-VPA available in the system for adsorption when the experiment was terminated (FIG. 2A). Diazepam adsorption also was restricted (approximately 65% of the total [¹⁴C]-DZP after 5 minutes of exposure) but did not appear to have reached its maximum adsorption in the presence of HSA (FIG. 2B). Digitoxin adsorption was limited at early time points of exposure but approached 100% adsorption after 5 minutes of DCC exposure in the presence of HSA (FIG. 2C).

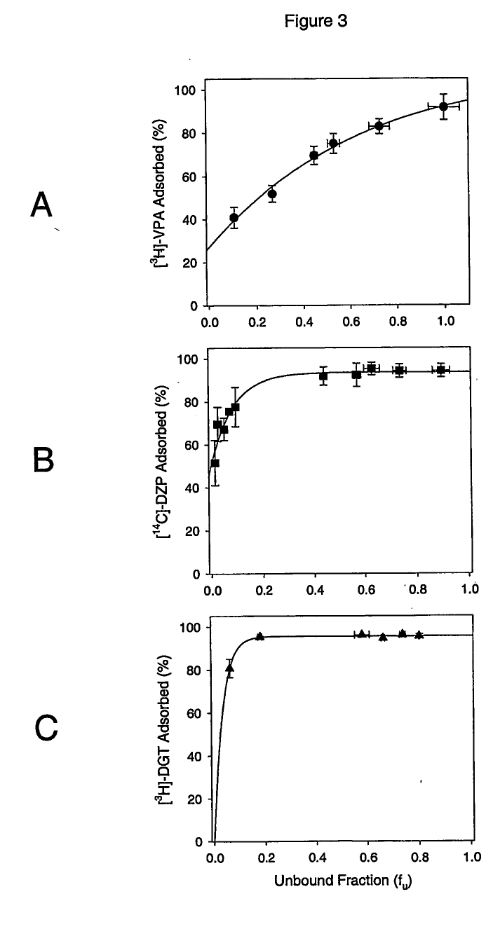

Extent of Ligand Adsorption as a Function of Unbound Fraction in the DCC Cartridge. FIGS. 3A-3B present graphs depicting a positive correlation between [³H]-VPA, [¹⁴C]-DZP and [³H]-DIGT adsorption to DCC with unbound fraction after 1 minute of charcoal exposure. The unbound fraction of ligand was manipulated by lowering the HSA concentration. The range of unbound fraction examined more substantially affected valproate adsorption (FIG. 3A) when compared to adsorption of the other two ligands. As unbound fraction increased and approached 0.2 for diazepam (FIG. 3B) and digitoxin (FIG. 3C), DCC adsorption was observed to be nearly complete.

The relationship between the extent of adsorption and the unbound fraction for each ligand can be described by Equation 1.
y=yₒ+a(1−e^{-bx}) Equation 1

wherein y is a value representing extent of adsorption, x is a value representing unbound fraction, yₒ is the intercept of the equation (extent of adsorption at an unbound fraction of 0), and a and b are empirical parameters, analogous to the slope in linear regression. Parameter estimates for curve fitting of all data simultaneously (n=3) for each of the site-specific ligands are presented in Table 4.

TABLE 4

| Ligand | Yo | a | b | r² |
|---|---|---|---|---|
| [³H]-VPA | 27.0 ± 4.1 | 82.9 ± 8.6 | 1.5 ± 0.4 | 0.98 |
| [¹⁴C]-DZP | 50.7 ± 4.9 | 42.9 ± 5.1 | 11.5 ± 2.9 | 0.93 |
| [³H]-DIGT | 0 | 95.6 ± 0.3 | 29.5 ± 0.7 | 0.99 |
| Multiple | 52.7 ± 8.4 | 41.6 ± 8.4 | 11.9 ± 4.6 | 0.78 |

^{a}Parameters presented as mean ± SE

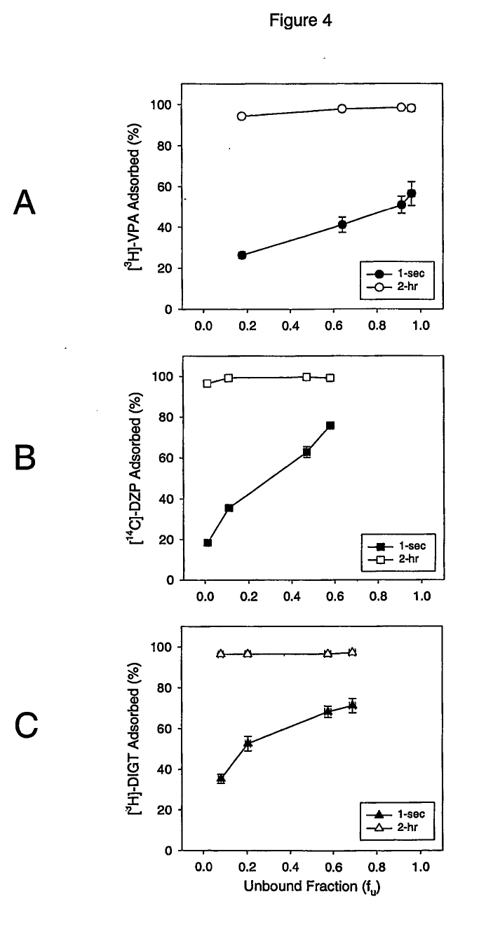

In addition to 1 minute of DCC exposure, the adsorption after 1 second and 2 hours was evaluated. As shown in FIGS. 4A-4C, the extent of DCC adsorption increased as unbound fraction increased for each ligand evaluated. After 2 hours of exposure to DCC, each ligand was adsorbed completely, independent of initial unbound fraction.

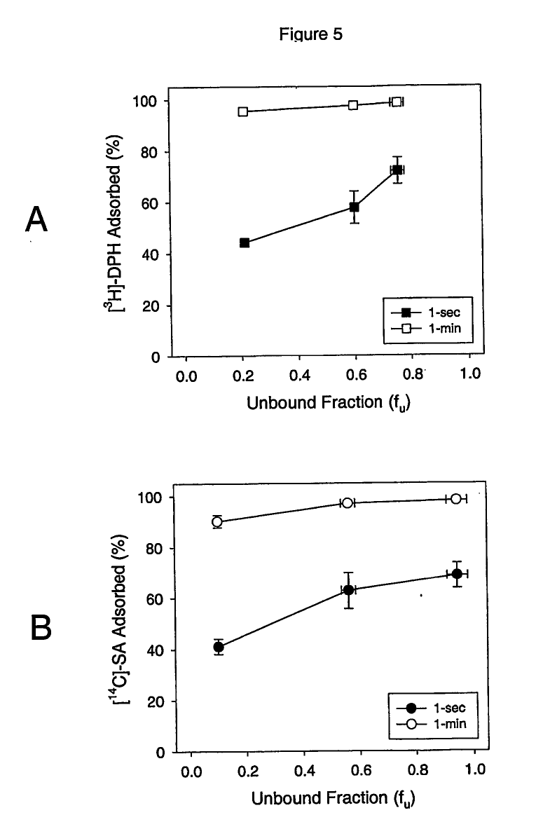

Alternative Ligand Evaluation. The extent of DCC adsorption as a function of unbound fraction for two alternative site I-specific ligands ([³H]-DPH and [¹⁴C]-SA) was evaluated. FIGS. 5A-5B are graphs depicting DCC adsorption after 1 second and 1 minute exposure for both [³H]-DPH and [¹⁴C]-SA was similar to the diazepam and digitoxin results. Adsorption was not restricted for either compound after 1 minute of exposure to DCC.

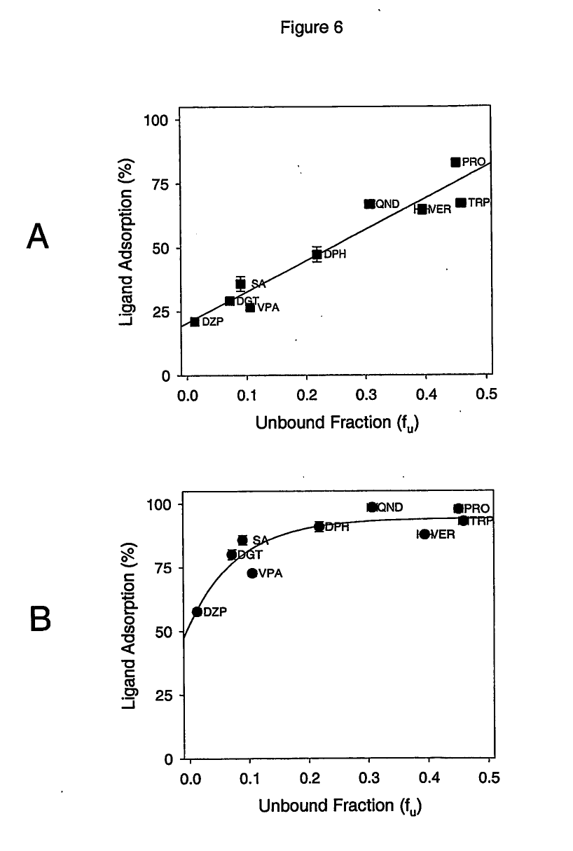

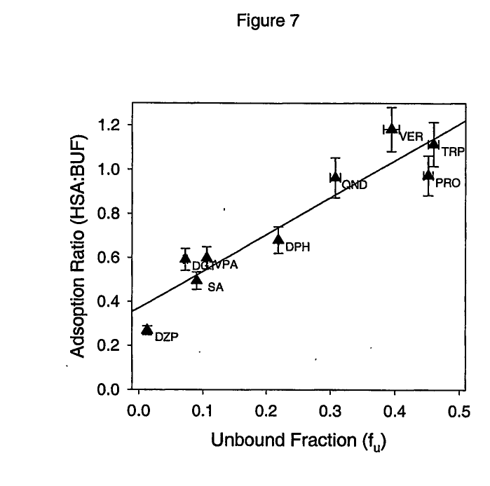

Multiple Ligand Correlation. FIGS. 6A and 6B present graphs depicting the extent of DCC adsorption of a set of compounds with a range of unbound fraction between 0.02 and 0.5. Ligand adsorption was observed to increase with unbound fraction in the DCC cartridge after 1 second (FIG. 6A) and 1 minute (FIG. 6B) of exposure in HSA-Buffer. The correlation (slope: 121.6, intercept: 20.5, r²=0.92) between the extent of charcoal adsorption and unbound fraction after 1 second of exposure was linear. After 1 minute exposure, curve fitting with Equation (1) revealed parameter estimates similar to those in determined for [¹⁴C]-DZP adsorption.

In order to evaluate the potential effect of differential ligand adsorption in the absence of HSA, 1-second exposure to DCC was performed in buffer for each ligand. Correcting the 1-second adsorption data in the presence of HSA for adsorption in the absence of HSA did not improve the correlation (slope: 1.7, intercept: 0.37, r²=0.89) between the extent of charcoal adsorption and unbound fraction.

### References

The references listed below as well as all references cited in the specification are incorporated herein by reference to the extent that they supplement, explain, provide a background for or teach methodology, techniques and/or compositions employed herein.
(1965) Darco Powdered and Granular Activated Carbon. Wilmington, Del.: Atlas Chemical Division, ICI America Inc.
Ascoli G, Bertucci C & Salvadori P (1995) Specific and competitive binding of drugs to human serum albumin: a difference circular dichroism approach. J Pharm Sci 84:737-741.
Ausubel F, ed (1995) Short Protocols in Molecular Biology, 3rd Edition. New York: Wiley.
Barré J, Chamouard J M, Houin G & Tillement JP (1985) Equilibrium dialysis, ultrafiltration, and ultracentrifugation compared for determining the plasma-protein-binding characteristics of valproic acid. Clin Chem 31:60-64.
Beaudry F, Le Blanc J C, Coutu M & Brown N K (1998) In vivo pharmacokinetic screening in cassette dosing experiments; the use of on-line Pprospekt liquid chromatography/atmospheric pressure chemical ionization tandem mass spectrometry technology in drug discovery. Rapid Commun Mass Spectrom 12:1216-1222.
Berman J, Haim K, Adkison K & Shaffer J (1997) Simultaneous pharmacokinetic screening of a mixture of compounds in the dog using API LC/MS/MS analysis for increased throughput. J Med Chem 40:827-829.
Cheremisinoff P & Morresi A (1978) Carbon Adsorption Applications. In: Carbon Adsorption Handbook (Cheremisinoff P & Ellerbusch F, eds), pp 1-53. Ann Arbor, Mich.: Ann Arbor Science Publishers.
Cooney D (1995) Fundamentals of activated Charcoal and the Adsorption Process. In: Activated Charcoal in Medical Applications (Cooney D, ed), pp 18-49. New York: Marcel Decker Inc.
Dagenais C, Koch A, Duttle A & Pollack G (1997) Evaluation of a Charcoal Adsorption Method for Determination of Drug Protein Binding. Pharm Res 14:S359.
Echizen H & Eichelbaum M (1986) Clinical pharmacokinetics of verapamil, nifedipine and diltiazem. Clin Pharmacokinet 11:425-449.
Frick L, Higton D, Wring S & Wells-Knect K (1998) Cassette Dosing: Rapid Estimation of in Vivo Pharmacokinetics. Medicinal Chemistry Research 8:472-477.
Furst D E, Tozer T N & Melmon K L (1979) Salicylate clearance, the resultant of protein binding and metabolism. Clin Pharmacol Ther 26:380-389.
Grasela T H, Sheiner L B, Rambeck B, Boenigk H E, Dunlop A, Mullen P W, Wadsworth J, Richens A, Ishizaki T, Chiba K & et al. (1983) Steady-state pharmacokinetics of phenytoin from routinely collected patient data. Clin Pharmacokinet 8:355-364.
Greenblatt D J, Allen M D, Harmatz J S & Shader R I (1980) Diazepam disposition determinants. Clin Pharmacol Ther 27:301-312.
Griffiths W C, Diamond I & Dextraze P (1975) The albumin binding of unconjugated bilirubin in serum. Clin Biochem 8:254-260.
Griffiths W J, Jonsson A P, Liu S, Rai D K & Wang Y (2001) Electrospray and tandem mass spectrometry in biochemistry. Biochem J 355:545-561.
Gruber H J, Kada G, Pragi B, Riener C, Hahn C D, Harms G S, Ahrer W, Dax T G, Hohenthanner K & Knaus H G (2000) Preparation of thiol-reactive Cy5 derivatives from commercial Cy5 succinimidyl ester. Bioconjug Chem 11:161-166.
Herbert V, Gottlieb C W, Lau K S, Gevirtz N R, Sharney L & Wasserman L R (1967) Coated charcoal assay of plasma iron binding capacity and iron using radioisotope dilution and hemoglobin-coated charcoal. J Nucl Med 8:529-541.
Huang J D & Oie S (1982) Effect of altered disopyramide binding on its pharmacologic response in rabbits. J Pharmacol Exp Ther 223:469-471.
Karas M, Bahr U & Dulcks T (2000) Nano-electrospray ionization mass spectrometry: addressing analytical problems beyond routine. Fresenius J Anal Chem 366:669-676.
Korfmacher W A, Cox K A, Ng K J, Veals J, Hsieh Y, Wainhaus S, Broske L, Prelusky D, Nomeir A & White R E (2001) Cassette-accelerated rapid rat screen: a systematic procedure for the dosing and liquid chromatography/atmospheric pressure ionization tandem mass spectrometric analysis of new chemical entities as part of new drug discovery. Rapid Commun Mass Spectrom 15:335-340.
Kowalski P & Stoerker J (2000) Accelerating discoveries in the proteome and genome with MALDI TOF MS. Pharmacogenomics 1:359-366.
Llopis M A, Granada M L, Audi L, Sanmarti A, Bel J, Sanchez-Planell L, Formiguera X, Marin F & Corominas A (1997) Analytical performance and clinical usefulness of two binding assays for growth hormone binding protein (GHBP) measurement: high performance liquid chromatography (HPLC)-gel filtration and dextran-coated charcoal adsorption. Clin Chim Acta 267:167-181.
Lowry O, Rosebrough N, Farr A & Randall R (1951) Protein Measurements with the Folin Phenol Reagent. J Biol Chem 193:265-275.
Maurer H H (2000) Screening procedures for simultaneous detection of several drug classes used for high throughput toxicological analyses and doping control. A review. Comb Chem High Throughput Screen 3:467-480.
McLoughlin D A, Olah T V & Gilbert J D (1997) A direct technique for the simultaneous determination of 10 drug candidates in plasma by liquid chromatography-atmospheric pressure chemical ionization mass spectrometry interfaced to a Prospekt solid-phase extraction system. J Pharm Biomed Anal 15:1893-1901.
McPherson M, Hames B & Taylor G, eds (1995) PCR 2: A Practical Approach. New York: IRL Press.
Mendel C M (1990) Rates of dissociation of sex steroid hormones from human sex hormone-binding globulin: a reassessment. J Steroid Biochem Mol Biol 37:251-255.
Mendel C M, Miller M B, Siiteri P K & Murai J T (1990) Rates of dissociation of steroid and thyroid hormones from human serum albumin. J Steroid Biochem Mol Biol 37:245-250.
Mingrone G, De Smet R, Greco A V, Bertuzzi A, Gandolfi A, Ringoir S & Vanholder R (1997) Serum uremic toxins from patients with chronic renal failure displace the binding of L-tryptophan to human serum albumin. Clin Chim Acta 260:27-34.
Mooradian A D (1988) Digitalis. An update of clinical pharmacokinetics, therapeutic monitoring techniques and treatment recommendations. Clin Pharmacokinet 15:165-179.
Newton R P, Bayliss M A, van Geyschem J, Harris F M, Games D E, Brenton G, Wilkins A C, Diffley P & Walton T J (1997) Kinetic analysis and multiple component monitoring of effectors of adenylyl cyclase activity by quantitative fast-atom bombardment mass spectrometry. Rapid Commun Mass Spectrom 11:1060-1066.
Ochs H R, Greenblatt D J & Woo E (1980) Clinical pharmacokinetics of quinidine. Clin Pharmacokinet5:150-168.
Olah T V, McLoughlin D A & Gilbert J D (1997) The simultaneous determination of mixtures of drug candidates by liquid chromatography/atmospheric pressure chemical ionization mass spectrometry as an in vivo drug screening procedure. Rapid Commun Mass Spectrom 11:17-23.
Pacifici G M & Viani A (1992) Methods of determining plasma and tissue binding of drugs. Pharmacokinetic consequences. Clin Pharmacokinet 23:449-468.
Parikh H H, McElwain K, Balasubramanian V, Leung W, Wong D, Morris M E & Ramanathan M (2000) A rapid spectrofluorimetric technique for determining drug-serum protein binding suitable for-high-throughput screening. Pharm Res 17:632-637.
Parrott M B & Barry M A (2000) Metabolic biotinylation of recombinant proteins in mammalian cells and in mice. Mol Ther 1:96-104.
Parrott M B & Barry M A (2001) Metabolic biotinylation of secreted and cell surface proteins from mammalian cells. Biochem Biophys Res Commun 281:993-1000.
Qin M, Nilsson M & Oie S (1994) Decreased elimination of drug in the presence of alpha-1-acid glycoprotein is related to a reduced hepatocyte uptake. J Pharmacol Exp Ther 269:1176-1181.
Riddell J G, Shanks R G & Brogden R N (1987) Celiprolol. A preliminary review of its pharmacodynamic and pharmacokinetic properties and its therapeutic use in hypertension and angina pectoris. Drugs 34:438-458.
Sambrook J & Russell D (2001) Molecular Cloning: A Laboratory Manual, 3rd Edition. Cold Spring Harbor, New York: Cold Spring Harbor Laboratory Press.
Suarez Varela A, Sandez Macho M I & Minones J (1992) Spectrofluorimetric study of the binding of 1-anilinonaphthalene-8-sulfonate to bovine serum albumin. J Pharm Sci 81:842-844.
Trung A H, Sirois G, Dube L M & McGilveray I J (1984) Comparison of the erythrocyte partitioning method with two classical methods for estimating free drug fraction in plasma. Biopharm Drug Dispos 5:281-290.
U.S. Pat. No. 5,545,895
U.S. Pat. No. 5,952,654
U.S. Pat. No. 5,118,937
U.S. Pat. No. 6,002,127
U.S. Pat. No. 6,017,693
U.S. Pat. No. 6,057,543
U.S. Pat. No. 6,093,300
U.S. Pat. No. 6,104,028
U.S. Pat. No. 6,107,623
Wahler D & Reymond J L (2001) Novel methods for biocatalyst screening. Curr Opin Chem Biol 5:152-158.
Walton T J, Bayliss M A, Pereira M L, Games D E, Genieser H G, Brenton A G, Harris F M & Newton R P (1998) Fast-atom bombardment tandem mass spectrometry of cyclic nucleotide analogues used as site-selective activators of cyclic nucleotide-dependent protein kinases. Rapid Commun Mass Spectrom 12:449-455.
White R & Manitpisitkul P (2001) Pharmacokinetic Theory of Cassette Dosing in Drug Discovery Screening. Drug Metabolism and Disposition 29:957-966.
Wong W H (1975) Determination of serum thyroxine after dissociation from thyroxine-binding globulin in alkaline solution and absorption on dextran-coated charcoal. Clin Chem 21:216-220.
Yuan J, Yang D C, Birkmeier J & Stolzenbach J (1995) Determination of protein binding by in vitro charcoal adsorption. J Pharmacokinet Biopharm 23:41-55.
Zaccara G, Messori A & Moroni F (1988) Clinical pharmacokinetics of valproic acid—1988. Clin Pharmacokinet 15:367-389.

It will be understood that various details of the invention can be changed without departing from the scope of the invention. Furthermore, the foregoing description is for the purpose of illustration only, and not for the purpose of limitation—the invention being defined by the claims appended hereto.

1. A method for evaluating binding of a ligand to a target protein, the method comprising:
(a) providing a sample comprising a target protein and a ligand, wherein the target protein and ligand are suspected to be bound reversibly together in a complex;
(b) preconditioning activated charcoal with the target protein;
(c) contacting the sample with the pre-conditioned activated charcoal for a time sufficient to allow for adsorption of unbound ligand to the activated charcoal;
(d) eluting the sample from the activated charcoal; and
(e) determining an amount of ligand in the eluted sample to thereby evaluate binding of the ligand to the target protein.

2. The method of claim 1 wherein the providing a sample comprises contacting a matrix comprising a target protein with at least one ligand for a time sufficient to allow for binding of the at least one ligand by the target protein.

3. The method of claim 2, wherein contacting a matrix comprising a target protein with at least one ligand comprises creating a suspension of the matrix comprising a target protein and the at least one ligand.

4. The method of claim 2, wherein the time sufficient to allow for binding comprises a duration equal to or less than about 30 minutes.

5. The method of claim 2, wherein the matrix comprises blood plasma.

6. The method of claim 5, wherein the blood plasma comprises human blood plasma.

7. The method of claim 1, wherein the target protein comprises serum albumin or α₁-acid-glycoprotein.

8. The method of claim 1, wherein the ligand comprises a chemical compound, a peptide, an oligonucleotide, a small molecule, or combinations thereof.

9. The method of claim 8, wherein the ligand is a candidate drug.

10. The method of claim 1, wherein the ligand further comprises a detectable label.

11. The method of claim 1, wherein the sample comprises a volume of about 200 μl.

12. The method of claim 1, wherein the preconditioning activated charcoal comprises contacting activated charcoal with the target protein or with a protein similar to the target protein for a time sufficient to allow for adsorption of the target protein or of the protein similar to the target protein to the activated charcoal.

13. The method of claim 12, wherein a time sufficient for adsorption of the target protein or of the protein similar to the target protein to the activated charcoal comprises about 1 second.

14. The method of claim 1, wherein the preconditioning comprises:
(a) preconditioning activated charcoal immediately prior to contacting the sample with the preconditioned activated charcoal; or
(b) preconditioning activated charcoal within 24 hours prior to contacting the sample with the preconditioned activated charcoal and rinsing the preconditioned activated charcoal immediately prior to contacting the sample with the preconditioned activated charcoal.

15. The method of claim 12, wherein preconditioning further comprises:
(a) applying the target protein or the protein similar to the target protein to a packed-bed activated charcoal cartridge, and
(b) eluting the target protein or the protein similar to the target protein from the packed-bed activated charcoal cartridge, whereby the activated charcoal is pre-conditioned.

16. The method of claim 15, wherein the applying the target protein or the protein similar to the target protein comprises providing a solution having a volume and a concentration -of the target protein or of the protein similar to the target protein, wherein the volume of the solution comprises a volume approximately equal to a volume of the sample, and wherein the concentration of the target protein or of the protein similar to the target protein in the solution comprises a concentration approximately equal to a concentration of the target protein in the sample.

17. The method of claim 1, wherein the activated charcoal comprises dextran-coated charcoal.

18. The method of claim 17, wherein the dextran-coated charcoal comprises dextrans having an average molecular weight of about 35 kDa to about 200 kDa.

19. The method of claim 18, wherein the dextran-coated charcoal comprises dextrans having an average molecular weight of about 50 kDa to about 150 kDa.

20. The method of claim 19, wherein the dextran-coated charcoal comprises dextrans having an average molecular weight of about 75 kDa to about 80 kDa.

21. The method of claim 17, wherein the dextran-coated charcoal comprises a fractional weight of about 10% to about 80% dextran.

22. The method of claim 21, wherein the dextran-coated charcoal comprises a fractional weight of about 10% to about 50% dextran.

23. The method of claim 22, wherein the dextran-coated charcoal comprises a fractional weight of about 10% dextran.

24. The method of claim 17, wherein the dextran-coated charcoal comprises a mass of about 5 mg to about 100 mg.

25. The method of claim 24, wherein the dextran-coated charcoal comprises a mass of about 5 mg to about 50 mg.

26. The method of claim 25, wherein the dextran-coated charcoal comprises a mass of about 20 mg.

27. The method of claim 1, wherein contacting the sample with pre-conditioned activated charcoal further comprises applying the sample to a packed-bed activated charcoal cartridge.

28. The method of claim 1, wherein the time sufficient to allow for adsorption of unbound ligand to the activated charcoal comprises about 1 second.

29. The method of claim 1, wherein eluting the sample comprises applying suction to the sample, whereby the sample is separated from the activated charcoal.

30. The method of claim 1, wherein determining an amount of ligand in the eluted sample comprises performing mass spectrometry analysis of the eluted sample.

31. The method of claim 10, wherein determining an amount of ligand in the eluted sample comprises detecting the detectably labeled ligand in the eluted sample.

32. A method for evaluating binding of a ligand to a target protein, the method comprising:
(a) providing a sample comprising a target protein and a first ligand, wherein the first ligand comprises a detectable label, and wherein the target protein and first ligand are suspected to be bound reversibly together in a complex;
(b) contacting the sample with a candidate second ligand for a time sufficient for displacement of the first ligand from the complex by the second ligand;
(c) preconditioning activated charcoal with the target protein;
(d) contacting the sample of (b) with the pre-conditioned activated charcoal for a time sufficient to allow for adsorption of unbound first ligand to the activated charcoal;
(e) eluting the sample from the activated charcoal; and
(f) determining an amount of first ligand in the eluted sample to thereby evaluate binding of the second ligand to the target protein.

33. The method of claim 32 wherein providing a sample comprises contacting a matrix comprising a target protein with a first ligand for a time sufficient to allow for binding of the first ligand by the target protein.

34. The method of claim 33, wherein contacting a matrix comprising a target protein with a first ligand comprises creating a suspension of the matrix comprising a target protein and the first ligand.

35. The method of claim 33, wherein the time sufficient to allow for binding comprises a duration equal to or less than about 30 minutes.

36. The method of claim 33, wherein the matrix comprises blood plasma.

37. The method of claim 36, wherein the blood plasma comprises human blood plasma.

38. The method of claim 32, wherein the sample comprises a volume of about 200 g.

39. The method of claim 32, wherein the target protein comprises serum albumin or α₁-acid-glycoprotein.

40. The method of claim 32, wherein the first ligand and the second ligand each comprise a chemical compound, a peptide, an oligonucleotide, a small molecule, or combinations thereof.

41. The method of claim 32, wherein the first ligand comprises a ligand that binds a specific binding site on a target protein.

42. The method of claim 41, further comprising determining an amount of the first ligand in the eluted sample to thereby evaluate binding of the second ligand to the specific ligand binding site of a target protein.

43. The method of claim 32, wherein the first ligand comprises a ligand that binds a plasma protein.

44. The method of claim 43, wherein the first ligand comprises a ligand that binds serum albumin or α₁-acid-glycoprotein.

45. The method of claim 44, wherein the first ligand comprises a ligand that binds site I, site II, or site III of human serum albumin.

46. The method of claim 45, wherein the first ligand comprises a site I-binding ligand selected from the group consisting of a cumarine and a pyrazolidine.

47. The method of claim 46, wherein the first ligand comprises a ligand selected from the group consisting of valproate, diphenylhydantoin, or salicylate.

48. The method of claim 45, wherein the first ligand comprises a site II-binding ligand selected from the group consisting of a benzodiazepine, an arylpropionate, and L-tryptophan.

49. The method of claim 48, wherein the first ligand comprises the site II-binding ligand diazepam.

50. The method of claim 45, wherein the first ligand comprises the site III-binding ligand digitoxin.

51. The method of claim 32 wherein the second ligand is a candidate drug.

52. The method of claim 32, wherein a time sufficient for displacement of the first ligand from the complex by the second ligand comprises a duration less than or equal to about 30 minutes.

53. The method of claim 32 wherein preconditioning activated charcoal comprises contacting activated charcoal with the target protein or with a protein similar to the target protein for a time sufficient to allow for adsorption of the target protein or the protein similar to the target protein to the activated charcoal.

54. The method of claim 53, wherein a time sufficient for adsorption of the target protein or of the protein similar to the target protein to the activated charcoal comprises about 1 second.

55. The method of claim 32, wherein the preconditioning comprises:
(c) preconditioning activated charcoal immediately prior to contacting the sample with the preconditioned activated charcoal; or
(d) preconditioning activated charcoal within 24 hours prior to contacting the sample with the preconditioned activated charcoal and rinsing the preconditioned activated charcoal immediately prior to contacting the sample with the preconditioned activated charcoal.

56. The method of claim 52, wherein preconditioning further comprises:
(a) applying the target protein or the protein similar to the target protein to a packed-bed activated charcoal cartridge, and
(b) eluting the target protein or the protein similar to the target protein from the packed-bed activated charcoal cartridge, whereby the activated charcoal is pre-conditioned.

57. The method of claim 56, wherein applying the target protein or the protein similar to the target protein comprises providing a solution having a volume and a concentration of the target protein or of the protein similar to the target protein, wherein the volume of the solution comprises a volume approximately equal to a volume of the sample, and wherein the concentration of the target protein or of the protein similar to the target protein in the solution comprises a concentration approximately equal to a concentration of the target protein in the sample.

58. The method of claim 32, wherein the activated charcoal comprises dextran-coated charcoal.

59. The method of claim 58, wherein the dextran-coated charcoal comprises dextrans having an average molecular weight of about 35 kDa to about 200 kDa.

60. The method of claim 59, wherein the dextran-coated charcoal comprises dextrans having an average molecular weight of about 50 kDa to about 150 kDa.

61. The method of claim 60, wherein the dextran-coated charcoal comprises dextrans having an average molecular weight of about 75 kDa to about 80 kDa.

62. The method of claim 58, wherein the dextran-coated charcoal comprises a fractional weight of about 10% to about 80% dextran.

63. The method of claim 62, wherein the dextran-coated charcoal comprises a fractional weight of about 10% to about 50% dextran.

64. The method of claim 63, wherein the dextran-coated charcoal comprises a fractional weight of about 10% dextran.

65. The method of claim 58, wherein the dextran-coated charcoal comprises a mass of about 5 mg to about 100 mg.

66. The method of claim 65, wherein the dextran-coated charcoal comprises a mass of about 5 mg to about 50 mg.

67. The method of claim 66, wherein the dextran-coated charcoal comprises a mass of about 20 mg.

68. The method of claim 32, wherein contacting the sample with pre-conditioned activated charcoal comprises applying the sample to a packed-bed activated charcoal cartridge.

69. The method of claim 32, wherein the time sufficient to allow for adsorption of unbound first ligand to the activated charcoal comprises about 1 second.

70. The method of claim 32, wherein eluting the sample comprises applying suction to the sample, whereby the sample is separated from the activated charcoal.

71. The method of claim 32, wherein determining an amount of ligand in the eluted matrix comprises detecting the detectable label of the first ligand in the eluted matrix.

72. A method for evaluating the susceptibility of a candidate drug to binding a protein found in the circulating blood of a warm-blooded vertebrate, the method comprising:
(a) providing a sample comprising a target protein and a ligand, wherein the ligand comprises a detectable label, and wherein the target protein and ligand are suspected to be bound reversibly together in a complex;
(b) contacting the sample with a candidate drug for a time sufficient for displacement of the ligand from the complex by the candidate drug;
(c) preconditioning activated charcoal with the target protein;
(d) contacting the sample of (b) with the pre-conditioned activated charcoal for a time sufficient to allow for adsorption of unbound ligand to the activated charcoal;
(e) eluting the sample from the activated charcoal; and
(f) determining an amount of ligand in the eluted sample to thereby evaluate the susceptibility of the candidate drug to binding a protein found in the circulating blood of a warm-blooded vertebrate.

73. The method of claim 72 wherein providing a sample comprises contacting a matrix comprising a target protein with a ligand for a time sufficient to allow for binding of the ligand by the target protein.

74. The method of claim 73, wherein contacting a matrix comprising a target protein with a ligand comprises creating a suspension of the matrix comprising a target protein and the ligand.

75. The method of claim 73, wherein the time sufficient to allow for binding comprises a duration equal to or less than about 30 minutes.

76. The method of claim 73, wherein the matrix comprises blood plasma.

77. The method of claim 76, wherein the blood plasma comprises human blood plasma.

78. The method of claim 72 wherein the sample comprises a volume of about 200 μl.

79. The method of claim 72, wherein the target protein comprises serum albumin or α₁-acid-glycoprotein.

80. The method of claim 72, wherein the ligand and the candidate drug each comprise a chemical compound, a peptide, an oligonucleotide, a small molecule, or combinations thereof.

81. The method of claim 72, wherein the first ligand comprises a ligand that binds a specific binding site on a target protein.

82. The method of claim 81, further comprising determining an amount of the first ligand in the eluted sample to thereby evaluate binding of the second ligand to the specific ligand binding site of a target protein.

83. The method of claim 72, wherein the ligand comprises a ligand that binds a plasma protein.

84. The method of claim 83, wherein the ligand comprises a ligand that binds serum albumin or α₁-acid-glycoprotein.

85. The method of claim 84, wherein the ligand comprises a ligand that binds site I, site II, or site III of human serum albumin.

86. The method of claim 85, wherein the ligand comprises a site I-binding ligand selected from the group consisting of a cumarine and a pyrazolidine.

87. The method of claim 86, wherein the ligand comprises a ligand selected from the group consisting of valproate, diphenylhydantoin, or salicylate.

88. The method of claim 85, wherein the ligand comprises a site II-binding ligand selected from the group consisting of a benzodiazepine, an arylpropionate, and L-tryptophan.

89. The method of claim 88, wherein the ligand comprises the site II-binding ligand diazepam.

90. The method of claim 85, wherein the ligand comprises the site III-binding ligand digitoxin.

91. The method of claim 72, wherein a time sufficient for displacement of the ligand from the complex by the candidate drug comprises a duration less than or equal to about 30 minutes.

92. The method of claim 72 wherein preconditioning activated charcoal comprises contacting activated charcoal with the target protein or with a protein similar to the target protein for a time sufficient to allow for adsorption of the target protein to the activated charcoal.

93. The method of claim 92, wherein a time sufficient for adsorption of the target protein or the protein similar to the target protein to the activated charcoal comprises about 1 second.

94. The method of claim 72, wherein the preconditioning comprises:
(e) preconditioning activated charcoal immediately prior to contacting the sample with the preconditioned activated charcoal; or
(f) preconditioning activated charcoal within 24 hours prior to contacting the sample with the preconditioned activated charcoal and rinsing the preconditioned activated charcoal immediately prior to contacting the sample with the preconditioned activated charcoal.

95. The method of claim 92, wherein preconditioning further comprises:
(a) applying the target protein or the protein similar to the target protein to a packed-bed activated charcoal cartridge; and
(b) eluting the target protein or the protein similar to the target protein from the packed-bed activated charcoal cartridge, whereby the activated charcoal is pre-conditioned.

96. The method of claim 95, wherein applying the target protein or the protein similar to the target protein comprises providing a solution having a volume and a concentration of the target protein or of the protein similar to the target protein, wherein the volume of the solution comprises a volume approximately equal to a volume of the sample, and wherein the concentration of the target protein or of the protein similar to the target protein in the solution comprises a concentration approximately equal to a concentration of the target protein in the sample.

97. The method of claim 72, wherein the activated charcoal comprises dextran-coated charcoal.

98. The method of claim 97, wherein the dextran-coated charcoal comprises dextrans having an average molecular weight of about 35 kDa to about 200 kDa.

99. The method of claim 98, wherein the dextran-coated charcoal comprises dextrans having an average molecular weight of about 50 kDa to about 150 kDa.

100. The method of claim 99, wherein the dextran-coated charcoal comprises dextrans having an average molecular weight of about 75 kDa to about 80 kDa.

101. The method of claim 97, wherein the dextran-coated charcoal comprises a fractional weight of about 10% to about 80% dextran.

102. The method of claim 101, wherein the dextran-coated charcoal comprises a fractional weight of about 10% to about 50% dextran.

103. The method of claim 102, wherein the dextran-coated charcoal comprises a fractional weight of about 10% dextran.

104. The method of claim 97, wherein the dextran-coated charcoal comprises a mass of about 5 mg to about 100 mg.

105. The method of claim 104, wherein the dextran-coated charcoal comprises a mass of about 5 mg to about 50 mg.

106. The method of claim 105, wherein the dextran-coated charcoal comprises a mass of about 20 mg.

107. The method of claim 72, wherein contacting the sample with pre-conditioned activated charcoal comprises applying the sample to a packed-bed activated charcoal cartridge.

108. The method of claim 72, wherein the time sufficient to allow for adsorption of unbound ligand to the activated charcoal comprises about 1 second.

109. The method of claim 72, wherein eluting the sample comprises applying suction to the sample, whereby the sample is separated from the activated charcoal.

110. The method of claim 72, wherein determining an amount of ligand in the eluted matrix comprises detecting the detectable label of the ligand in the eluted matrix.

111. A packed-bed charcoal cartridge for evaluating ligand binding to a target protein, comprising:
(a) a column comprising a sample chamber, a sample addition port, and a sample elution port adapted for fluid/gaseous communication with a suction source; and
(b) an activated charcoal packed-bed positioned between the sample chamber and the sample elution port, wherein the charcoal packed-bed is in fluid/gaseous communication with the sample chamber and with the sample elution port.

112. The packed-bed charcoal cartridge of claim 111, wherein the column comprises a sample chamber capable of holding about one (1) milliliter of liquid volume.

113. The packed-bed charcoal cartridge of claim 112, wherein the column comprises a PREPSEP® column, and wherein a bottom of the sample chamber comprises a frit.

114. The packed-bed charcoal cartridge of claim 111, wherein the activated charcoal comprises dextran-coated charcoal.

115. The packed-bed charcoal cartridge of claim 114, wherein the dextran-coated charcoal comprises dextrans having an average molecular weight of about 35 kDa to about 200 kDa.

116. The packed-bed charcoal cartridge of claim 115, wherein the dextran-coated charcoal comprises dextrans having an average molecular weight of about 50 kDa to about 150 kDa.

117. The packed-bed charcoal cartridge of claim 116, wherein the dextran-coated charcoal comprises dextrans having an average molecular weight of about 75 kDa to about 80 kDa.

118. The packed-bed charcoal cartridge of claim 114, wherein the dextran-coated charcoal comprises a fractional weight of about 10% to about 80% dextran.

119. The packed-bed charcoal cartridge of claim 118, wherein the dextran-coated charcoal comprises a fractional weight of about 10% to about 50% dextran.

120. The packed-bed charcoal cartridge of claim 119, wherein the dextran-coated charcoal comprises a fractional weight of about 10% dextran.

121. The packed-bed charcoal cartridge of claim 114, wherein the dextran-coated charcoal comprises a mass of about 5 mg to about 100 mg.

122. The packed-bed charcoal cartridge of claim 121, wherein the dextran-coated charcoal comprises a mass of about 5 mg to about 50 mg.

123. The packed-bed charcoal cartridge of claim 121, wherein the dextran-coated charcoal comprises a mass of about 20 mg.

124. The packed-bed charcoal cartridge of claim 111 further comprising a filter positioned adjacent to and below the frit.

125. The packed-bed charcoal cartridge of claim 124 wherein the filter comprises a 1-cm glass filter.

126. An apparatus for high-throughput analysis of ligand binding to a protein comprising an array of packed-bed activated charcoal cartridge units of claim 111.

127. The apparatus of claim 126, wherein the array comprises 96 packed-bed activated charcoal cartridge units or an integer multiple thereof.

128. A method for preparing a packed-bed activated charcoal cartridge unit, the method comprising:
(a) providing a column comprising a sample chamber, a sample addition port, a sample elution port adapted for fluid/gaseous communication with a suction source, and a barrier positioned between the sample chamber and the sample elution port;
(b) applying activated charcoal in a liquid suspension to the column; and
(c) eluting the liquid from the column, whereby the activated charcoal is packed adjacent to the barrier, and whereby a packed-bed activated charcoal cartridge is prepared.

129. The method of claim 128, wherein the column comprises a sample chamber capable of holding a liquid volume comprising about one (1) milliliter.

130. The method of claim 129, wherein the column comprises a PREPSEP® column, and wherein the barrier comprises a frit.

131. The method of claim 128, wherein the activated charcoal comprises dextran-coated charcoal.

132. The method of claim 131, wherein the dextran-coated charcoal comprises dextrans having an average molecular weight of about 35 kDa to about 200 kDa.

133. The method of claim 131, wherein the dextran-coated charcoal comprises dextrans having an average molecular weight of about 55 kDa to about 150 kDa.

134. The method of claim 133, wherein the dextran-coated charcoal comprises dextrans having an average molecular weight of about 75 kDa to about 80 kDa.

135. The method of claim 131, wherein the dextran-coated charcoal comprises a fractional weight of about 10% to about 80% dextran.

136. The method of claim 135, wherein the dextran-coated charcoal comprises a fractional weight of about 10% to about 50% dextran.

137. The method of claim 136, wherein the dextran-coated charcoal comprises a fractional weight of about 10% dextran.

138. The method of claim 131, wherein the dextran-coated charcoal comprises a mass of about 5 mg to about 100 mg.

139. The method of claim 138, wherein the dextran-coated charcoal comprises a mass of about 5 mg to about 50 mg.

140. The method of claim 139, wherein the dextran-coated charcoal comprises a mass of about 20 mg.

141. The method of claim 138, further comprising providing a filter to the column positioned adjacent to and below the barrier to prevent elution of the activated charcoal from the column.

142. The method of claim 141, wherein the filter comprises a 1-cm glass filter.
